(19) United States
(12) Patent Application Publication
(10) Pub. No.: US 2023/0157941 A1
(43) Pub. Date: May 25, 2023

(54) COMPOSITIONS BASED ON AT LEAST TWO GLYCOSAMINOGLYCANS

(71) Applicant: TEOXANE SA, Geneve (CH)

(72) Inventor: François BOURDON, Gaillard (FR)

(21) Appl. No.: 17/916,896

(22) PCT Filed: Apr. 12, 2021
(86) PCT No.: PCT/EP2021/059432

§ 371 Date: Oct. 4, 2022

(30) Foreign Application Priority Data
Apr. 10, 2020 (FR) FR2003628

(51) Int. Cl. A61K 8/73; A61K 8/04; A61L 27/20; A61L 27/52; A61Q 19/08; C08J 3/075; C08L 5/08; C08J 3/24

(52) U.S. Cl. CPC A61K 8/735; A61K 8/042; A61L 27/20; A61L 27/52; A61Q 19/08; C08J 3/24; C08J 3/075; C08L 5/08; A61K 2800/91; A61L 2400/06; A61L 2430/34; C08J 2305/08

(57) ABSTRACT

A hyaluronic acid-based aqueous gel including crosslinked hyaluronic acid and from 30 to 70% by mass with respect to the total mass of hyaluronic acid, of a water-soluble hyaluronic acid. A method for preparing an aqueous gel having at least a crosslinked hyaluronic acid and at least an uncrosslinked hyaluronic acid. Also an aqueous gel obtainable by the method compositions, a cosmetic and/or dermatological composition including the aqueous gels, and a kit comprising a pre-filled syringe comprising the aqueous gel or said composition. These gels and compositions are useful in particular for treating an alteration in the surface appearance of the skin and/or for augmenting and/or filling soft tissue.

### TECHNICAL FIELD

The present invention relates to a hyaluronic acid-based aqueous gel comprising at least crosslinked hyaluronic acid and at least one type of water-soluble hyaluronic acid. The present invention further relates to a method for preparing supplemented compositions based on glycosaminoglycans, in particular on hyaluronic acids, that are soft, stable, sterile and injectable. The aqueous gel and compositions thus obtained comprise at least two types of glycosaminoglycans, including at least one crosslinked glycosaminoglycan, in particular at least one crosslinked hyaluronic acid, prepared from a non-crosslinked high-molar-mass glycosaminoglycan and at least one non-crosslinked low-molar-mass glycosaminoglycan, in particular prepared from at least one non-crosslinked high-molar-mass hyaluronic acid and at least one non-crosslinked low-molar-mass hyaluronic acid.

### PRIOR ART

Polysaccharides, such as glycosaminoglycans, are widely used in the biomedical field. In particular, most products marketed for aesthetic and/or cosmetic applications are based on hyaluronic acid.

To improve the quality of the skin, unmodified hyaluronic acid gels are attractive because they have the advantage of being perfectly biocompatible. However, after implantation in vivo, they degrade very rapidly. Thus, the effects of such gels are only of short duration.

In order to increase its durability in vivo and its resistance to degradation, hyaluronic acid is usually modified by crosslinking. Crosslinked hyaluronic acid gels can be obtained by different preparation processes. Polysaccharides, such as hyaluronic acid, can be crosslinked with different crosslinking agents, such as epoxy agents, aldehydes like glutaraldehyde, divinyl sulfone (DVS) or polyamines. Currently, the most common agent used to crosslink hyaluronic acid is an epoxy agent, 1,4-butanediol diglycidyl ether (BDDE).

These crosslinking processes require two main steps, namely hydration of the polysaccharide and crosslinking.

The commercial products based on hyaluronic acid thus prepared generally comprise, and according to their indications, a fraction of crosslinked hyaluronic acid and a modification degree ranging from 1% to 10%.

Nevertheless, the increase in the crosslinking of hyaluronic acid results in the preparation of a more chemically modified gel, which is therefore potentially less biocompatible. On the other hand, the mechanical performances of these compositions are limited and result in a very elastic and/or brittle final product.

However, for reasons of product safety and performance, there is an interest in having durable in vivo compositions comprising a biocompatible polysaccharide, as natural and as little modified, thus as little crosslinked, as possible.

In addition to the degree of crosslinking, the concentration of the glycosaminoglycan used is a key parameter in determining the mechanical and resistance properties of a composition. Thus, an alternative to increase the in vivo durability and the degradation resistance of a polysaccharide-based composition is to increase its total concentration in the final composition. However, in addition to increasing its resistance and its durability, the increase in the total concentration of said glycosaminoglycan results in a thickening of the composition, with a significant increase in its viscosity. This increase in viscosity thus makes the gel difficult to inject and unable to integrate naturally into the tissues. In particular, the increase in the total hyaluronic acid concentration is known to be the source of the occurrence of post-injection papules.

Thus, for all these reasons, most crosslinked hyaluronic acid products marketed to date have a total hyaluronic acid concentration of only about 25 or 26 mg/g. However, although potentially useful for volumizing dermal filling applications, such gels are not suitable for cutaneous applications of regeneration and improvement of the appearance of the skin in which mechanical filling is not the main effect sought and which require an injection in the superficial zones of the skin. For the latter type of injection, it is in fact desirable to prepare a gel with lower elastic (G′) and viscous (G″) moduli, that is soft, easy to inject and diffuses easily into the tissues.

Still in this context of increasing the resistance and persistence of products based on glycosaminoglycans, such as hyaluronic acid, it has also been proposed to add various compounds with protective properties with respect to hyaluronic acid. Thus, against the degradation of hyaluronic acid under heat, the application WO2014/032804 proposes the addition of magnesium ascorbyl phosphate, the application WO2013/186493 that of sucrose octasulfate and the application WO2007/077399 of glycerol. However, the addition of this type of stabilizing compounds increases the uncertainties regarding the biocompatibility of the products.

Consequently, there remains a need for sterile gels based on glycosaminoglycans, and more particularly on hyaluronic acid, that are soft, easy to inject and diffuse easily into the tissues, while being sufficiently durable and effective for regeneration and/or improvement of the appearance of the skin.

In particular, there remains a need to increase the resistance and the durability of compositions based on glycosaminoglycans, such as hyaluronic acid, to overcome the problems of the prior art. The present invention proposes to meet these needs.

### SUMMARY OF THE INVENTION

According to a first aspect, the present invention relates to a hyaluronic acid-based aqueous gel comprising crosslinked hyaluronic acid, characterized in that said gel comprises at least:
a water-soluble hyaluronic acid with a mass-average molar mass ranging from 0.02 MDa to 0.30 MDa, called “sHA LMW”, in an amount of from 30% to 70% by mass with respect to the total mass of hyaluronic acid; and
optionally, a water-soluble hyaluronic acid with a mass-average molar mass greater than 0.30 MDa, called “sHA HMW”,
and in that said water-soluble hyaluronic acids have a modification degree less than or equal to 5%, preferentially less than or equal to 3%, more preferentially less than or equal to 1%, respective percentages of soluble hyaluronic acids being determined by diluting the aqueous gel within a pH 7.2 solution of 150 nM sodium nitrate containing 0.02 % NaN₃ at 25° C. for 5 days and centrifugating it at 4400 rpm for 10 minutes and filtering at 0.45 mm such diluted gel to obtain a filtrate then analysing said filtrate with a size exclusion chromatography instrument equipped with a multiangle light scattering (MALS) detector and a refractive index (RI) detector with a refractive index increment (dn/dc) set at 0.165 mL/g and a liquid chromathography pumping station equipped with a dual set of size exclusion columns adapted for molecules with mass-average molar mass comprised from 0.001 Da to 20 MDa, with a pH 7.2 mobile phase of sodium nitrate at a flow rate of 0.3 mL/min.

The present text describes a process for preparing an aqueous gel comprising at least one crosslinked glycosaminoglycan and at least one non-crosslinked glycosaminoglycan, said process comprising at least the steps consisting in:
a) having an aqueous solution comprising at least one crosslinked glycosaminoglycan called “crosslinked glycosaminoglycan HMW” formed from non-crosslinked glycosaminoglycan of molar mass greater than or equal to 0.50 MDa, preferentially greater than 1.00 MDa, more preferentially ranging from 1.00 MDa to 4.00 MDa, and from a crosslinking agent;
b) having an aqueous solution comprising at least one non-crosslinked glycosaminoglycan of molar mass ranging from 0.04 MDa to 0.30 MDa, preferentially from 0.08 MDa to 0.20 MDa, more preferentially from 0.08 MDa to 0.15 MDa, still more preferentially from 0.08 MDa to 0.10 MDa, called “non-crosslinked glycosaminoglycan LMW” ; and,
c) forming a homogeneous mixture of all or part of said solutions from steps a) and b).

According to a second aspect, the present invention relates to a process for preparing an aqueous gel comprising at least one crosslinked hyaluronic acid and at least one non-crosslinked hyaluronic acid, said process comprising at least the steps consisting in:
a) having an aqueous solution comprising at least one crosslinked hyaluronic acid, called “crosslinked HA HMW”, formed from a non-crosslinked hyaluronic acid of mass-average molar mass greater than or equal to 0.50 MDa, preferentially greater than 1.00 MDa, more preferentially ranging from 1.00 MDa to 4.00 MDa, and a crosslinking agent;
b) having an aqueous solution comprising at least one non-crosslinked hyaluronic acid, called “non-crosslinked HA LMW”, of mass-average molar mass ranging from 0.04 MDa to 0.30 MDa, preferentially from 0.08 MDa to 0.20 MDa; more preferentially from 0.08 MDa to 0.15 MDa ; and,
c) forming a homogeneous mixture of all or part of said solutions of steps a) and b); characterized in that it comprises the use of hyaluronic acid in a mass ratio of “non-crosslinked HA LMW” to “HA HMW” of 1:3 to 2:1.

Unexpectedly, the inventors have found that supplementation of compositions comprising at least one crosslinked glycosaminoglycan prepared from at least one non-crosslinked high-molar-mass glycosaminoglycan, with at least one non-crosslinked low-molar-mass glycosaminoglycan, in particular the supplementation of compositions comprising at least a crosslinked hyaluronic acid prepared from at least one non-crosslinked high-molar-mass hyaluronic acid with at least one non-crosslinked low-molar-mass hyaluronic acid, amplifies the resistance of the final composition.

As shown in the examples below, the result is:
improved resistance to degradation compared with the same composition without added non-crosslinked low-molar-mass glycosaminoglycan and more particularly without added non-crosslinked low-molar-mass hyaluronic acid; and
better resistance to degradation compared with compositions with the same total concentration of glycosaminoglycan, more particularly of hyaluronic acid, and the same degree of crosslinking of the fraction of crosslinked glycosaminoglycan, more particularly of crosslinked hyaluronic acid.

Due to this improved resistance to degradation, the compositions according to the invention are more resistant to heat sterilization. Thus, between two sterile and injectable compositions of similar rheological properties before sterilization, one in accordance with the invention and the other prepared according to the teachings of the prior art, the former shows rheological properties after sterilization superior to those of the latter.

Thanks to their increased resistance to degradation, the compositions according to the invention are advantageously capable of withstanding variable temperatures, in particular during their transport and storage.

Furthermore, supplementation with at least one non-crosslinked low-molar-mass glycosaminoglycan, in particular with at least one non-crosslinked low-molar-mass hyaluronic acid, makes it possible to prepare soft, easy-to-inject and easily diffusing compositions with an increased total glycosaminoglycan concentration, thus with one or more significant biomechanical and/or biological effects on the physiology of the skin.

Moreover, the use of a composition prepared according to the invention allows the simultaneous administration of glycosaminoglycans, in particular hyaluronic acids, of different molar masses conducive to a diversification of the physiological effects of the glycosaminoglycan, in particular of the hyaluronic acid, at the treated site.

According to a third aspect, the present invention relates to an aqueous gel of glycosaminoglycans, in particular of hyaluronic acids, obtainable according to a process in accordance with the invention.

According to a fourth aspect, the present invention relates to a cosmetic and/or dermatological composition comprising an aqueous gel as defined in the present invention.

The present invention also concerns compositions obtained according to a method in accordance with the invention.

The invention also relates to an aqueous gel according to the invention or a composition according to the invention for use in the prevention and/or treatment of an alteration of the viscoelastic or biomechanical properties of the skin.

The invention also relates to an aqueous gel according to the invention or a dermatological composition according to the invention for use in the prevention and/or treatment of an alteration in the surface appearance of the skin, for instance induced by external factors such as stress, atmospheric pollution, tobacco or prolonged exposure to ultraviolet (UV) light.

Preferentially, the invention relates to an aqueous gel according to the invention or a dermatological composition according to the invention for use in soft tissue augmentation and/or filling.

It also relates to a non-therapeutic cosmetic use of an aqueous gel as defined in the present invention or a cosmetic composition as defined in the present invention for preventing and/or treating an alteration in the surface appearance of the skin, for instance of cutaneous signs of chronological aging.

It also relates to a non-therapeutic cosmetic use of an aqueous gel as defined in the present invention or a cosmetic composition as defined in the present invention for augmenting and/or filling soft tissue, in particular filling wrinkles.

### BRIEF DESCRIPTION OF THE DRAWINGS

FIG. 1 is a graphical representation of the loss of elastic modulus (G′) upon autoclave sterilization (ΔG′(%)), the concentration of non-crosslinked low-molar-mass hyaluronic acid, and the concentration of crosslinked and non-crosslinked high-molar-mass hyaluronic acid of samples 1-1 to 1-3.

FIG. 2 is a graph showing the losses of elastic modulus (G′) upon autoclave sterilization (ΔG′(%)) of a sample 3-5 without added non-crosslinked hyaluronic acid and of samples 3-6 to 3-9 comprising added non-crosslinked hyaluronic acid of molar mass 0.04 MDa, 0.10 MDa, 0.20 MDa and 1.50 MDa, respectively.

FIG. 3 is a graph showing the percentages of improvement in loss of elastic modulus (G′) upon autoclave sterilization of a sample 3-5 without added non-crosslinked hyaluronic acid and of samples 3-6 to 3-9 comprising added non-crosslinked hyaluronic acid of molar mass 0.04 MDa, 0.10 MDa, 0.20 MDa and 1.50 MDa, respectively.

FIG. 4 is a graphical representation of the loss of elastic modulus (G′) upon autoclave sterilization (ΔG′(%)), the concentration of non-crosslinked low-molar-mass hyaluronic acid, and the concentration of crosslinked and non-crosslinked high-molar-mass hyaluronic acid of a sample 4-1 (not supplemented) and of samples 4-2 to 4-4.

FIG. 5 is a graph showing the differences in elastic modulus (G′) before and after hyaluronidase treatment (ΔG′(%)) of a sample 6-1 without added non-crosslinked hyaluronic acid and of samples 6-2 and 6-3 comprising added non-crosslinked hyaluronic acid of molar mass 0.10 MDa and 1.50 MDa, respectively.

### DETAILED DESCRIPTION

### Definition of Terms According to the Invention

“Glycosaminoglycans” are defined as long chains composed of repeating disaccharide units in which one of the two carbohydrate residues is an amino carbohydrate (N-acetylglucosamine or N-acetylgalactosamine) and the second a uronic (glucuronic or iduronic) acid or a galactose. Glycosaminoglycans include hyaluronic acid, heparosan, chondroitin sulfate, dermatan sulfate and keratan sulfate. A particularly preferred glycosaminoglycan is hyaluronic acid.
“Hyaluronic acid” means hyaluronic acid, or hyaluronan, and its derivatives. Consequently, the term “hyaluronic acid” also includes hyaluronic acid salts such as sodium hyaluronate and hyaluronic acids that have been chemically modified, for example by oxidation, reduction, deacetylation, sulfation or amidation. Hyaluronic acid is a linear polysaccharide with alternating D-glucuronic acid and N-acetyl-D-glucosamine units linked by alternating β-1,3 glycosidic and β-1,4 glycosidic bonds.

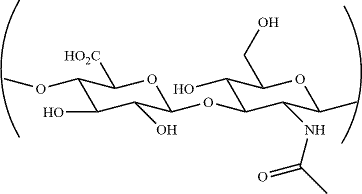

It is naturally present in several tissues of the human body and is degraded by enzymes, hyaluronidases, also present in the body, and/or via oxidation mechanisms.
A “water-soluble hyaluronic acid” or “soluble hyaluronic acid” or “extractable hyaluronic acid”, also called “sHA”, means a hyaluronic acid that can be extracted from a hyaluronic acid-based gel when the latter is swollen in an excess of aqueous medium. The respective percentages of soluble hyaluronic acids being determined by diluting the aqueous gel within a pH 7.2 solution of 150 nM sodium nitrate containing 0.02 % NaN₃ at 25° C. for 5 days and centrifugating it at 4400 rpm for 10 minutes and filtering at 0.45 mm such diluted gel to obtain a filtrate then analysing said filtrate with a size exclusion chromatography instrument equipped with a multiangle light scattering (MALS) detector and a refractive index (RI) detector with a refractive index increment (dn/dc) set at 0.165 mL/g and a liquid chromathography pumping station equipped with a dual set of size exclusion columns adapted for molecules with mass-average molar mass comprised from 0.001 Da to 20 MDa, with a pH 7.2 mobile phase of sodium nitrate at a flow rate of 0.3 mL/min. Practically, the concentration of gel in the mobile phase for the dilution step as well as the injected volume in the SEC are adequately adjusted by the person skilled in the art as a function of the supernatant composition to max out the signal-to-bruise ratio and avoid column overload. Conversely, a “non-water-soluble hyaluronic acid” or non-soluble hyaluronic acid” or “non-extractable hyaluronic acid”, also called “nsHA”, means a hyaluronic acid which cannot be extracted from a hyaluronic acid-based gel when the latter is swollen in an excess of aqueous medium.
The “mass average molar mass”, expressed in g/mol or Da and abbreviated Mw, of a glycosaminoglycan, in particular hyaluronic acid, refers to the mass-average molar mass of the glycosaminoglycan molecules used. The mass-average molar mass of a glycosaminoglycan can be determined by various methods known to the person skilled in the art, such as by capillary electrophoresis, by size exclusion chromatography, or from a measurement of intrinsic viscosity. In the present text, the expressions “molar mass”, “average molar mass”, “mass-average molar mass” or “mass molar mass” may be used interchangeably.
The “intrinsic viscosity,” ([η]), expressed in mL/g or m³/kg, of a glycosaminoglycan, in particular hyaluronic acid, refers to the contribution of the glycosaminoglycan to the viscosity of a solution. It can be measured by flow through a capillary viscometer. It represents the hydrodynamic specific volume of the polymer. The lower the intrinsic viscosity of a glycosaminoglycan, the lower its molar mass. For a given glycosaminoglycan, the intrinsic viscosity is related to the molar mass by an empirical rotation, the Mark-Houwink relation, which can be understood by any person skilled in the art, and of which the formula is the following:
$$[ η ] = K \times M {w}^{α}$$
with:
   K a constant function of the polymer studied, the solvent used and the temperature, in particular, K is related to the dimensions of the chains of the polymer studied;
   α a constant function of the polymer studied, the solvent used and the temperature, in particular, α is related to the conformation of the polymer studied; and,
   Mw the mass-average molar mass of the polymer, expressed in daltons (Da).
“Low-molar-mass glycosaminoglycan”, also called “glycosaminoglycan LMW” or “LMW glycosaminoglycan”, means a glycosaminoglycan, as a raw material, with a mass-average molar mass ranging from 0.04 to 0.30 MDa, preferentially from 0.08 MDa to 0.20 MDa, more preferentially from 0.08 MDa to 0.15 MDA, still more preferentially from 0.08 MDa to 0.10 MDa.
“Low-molar-mass hyaluronic acid”, also called “hyaluronic acid LMW” or “HA LMW” or “LMW hyaluronic acid” or “LMW HA”, means a hyaluronic acid, as a raw material, of molar mass ranging from 0.04 to 0.30 MDa, preferentially from 0.08 to 0.20 MDa, more preferentially from 0.08 to 0.15 MDa which corresponds to a hyaluronic acid of low intrinsic viscosity, i.e., of intrinsic viscosity ranging from 0.10 to 0.8 m³/kg preferably from 0.2 to 0.5 m³/kg, more preferably from 0.2 to 0.4 m³/kg measured according to the method indicated by the European Pharmacopoeia (edition 01/2017:1472).
“Low-molar-mass water soluble hyaluronic acid” or “Low-molar-mass soluble hyaluronic acid”, also called “sHA LMW” or “LMW sHA”, means a soluble hyaluronic acid of molar mass ranging from 0.02 MDa to 0.30 MDa, preferentially from 0.04 MDa to 0.20 MDa, more preferentially from 0.04 MDa to 0.15 MDa, measured on the final gel according to the method detailed in claim 1, that is to say, by diluting the aqueous gel within a pH 7.2 solution of 150 nM sodium nitrate containing 0.02 % NaN₃ at 25° C. for 5 days and centrifugating it at 4400 rpm for 10 minutes and filtering at 0.45 mm such diluted gel to obtain a filtrate then analysing said filtrate with a size exclusion chromatography instrument equipped with a multiangle light scattering (MALS) detector and a refractive index (RI) detector with a refractive index increment (dn/dc) set at 0.165 mL/g and a liquid chromathography pumping station equipped with a dual set of size exclusion columns adapted for molecules with mass-average molar mass comprised from 0.001 Da to 20 MDa, with a pH 7.2 mobile phase of sodium nitrate at a flow rate of 0.3 mL/min. Practically, the concentration of gel in the mobile phase for the dilution step as well as the injected volume in the SEC are adequately adjusted by the person skilled in the art as a function of the supernatant composition to max out the signal-to-bruise ratio and avoid column overload.
“High-molar-mass glycosaminoglycan”, also called “glycosaminoglycan HMW” or “HMW glycosaminoglycan”, means a glycosaminoglycan, as a raw material, of molar mass greater than 0.50 MDa, preferably greater than 1.00 MDa, and more preferably ranging from 1.00 MDa to 4.00 MDa.
“High-molar-mass hyaluronic acid”, also called “hyaluronic acid HMW” or “HA HMW” or “HMW hyaluronic acid” or “HMW HA”, means a hyaluronic acid, as a raw material, of molar mass greater than 0.50 MDa, preferably greater than 1.00 MDa, and more preferably ranging from 1.00 MDa to 4.00 MDa, which corresponds to a hyaluronic acid of high intrinsic viscosity, i.e., of intrinsic viscosity greater than 0.8 m³/kg, preferably greater than 1.2 m³/kg, more preferably ranging from 1.2 m³/kg to 3.5 m³/kg, measured according to the method indicated by the European Pharmacopoeia (edition 01/2017: 1472).
“High-molar-mass soluble hyaluronic acid”, or “High-molar-mass water-soluble hyaluronic acid” also called “sHA HMW” or “HMW sHA”, means a soluble hyaluronic acid of molar mass higher than 0.30 MDa, preferentially higher than 0.30 MDa and lower than or equal to 4.00 MDa, measured on the final gel according to the method detailed in claim 1, that is to say, by diluting the aqueous gel within a pH 7.2 solution of 150 nM sodium nitrate containing 0.02 % NaN₃ at 25° C. for 5 days and centrifugating it at 4400 rpm for 10 minutes and filtering at 0.45 mm such diluted gel to obtain a filtrate then analysing said filtrate with a size exclusion chromatography instrument equipped with a multiangle light scattering (MALS) detector and a refractive index (RI) detector with a refractive index increment (dn/dc) set at 0.165 mL/g and a liquid chromathography pumping station equipped with a dual set of size exclusion columns adapted for molecules with mass-average molar mass comprised from 0.001 Da to 20 MDa, with a pH 7.2 mobile phase of sodium nitrate at a flow rate of 0.3 mL/min. Practically, the concentration of gel in the mobile phase for the dilution step as well as the injected volume in the SEC are adequately adjusted by the person skilled in the art as a function of the supernatant composition to max out the signal-to-bruise ratio and avoid column overload.
A “crosslinked glycosaminoglycan” refers to a glycosaminoglycan modified with a crosslinking agent during a crosslinking reaction. Conversely, a “non-crosslinked glycosaminoglycan” refers to a glycosaminoglycan that has not been modified with a crosslinking agent and therefore has not undergone a crosslinking reaction.
A “crosslinked hyaluronic acid” refers to a hyaluronic acid modified with a crosslinking agent during a crosslinking reaction. Conversely, a “non-crosslinked hyaluronic acid” refers to a hyaluronic acid that has not been modified with a crosslinking agent and has therefore not undergone a crosslinking reaction.
Non-crosslinked» and «uncrosslinked » are synonymous terms and may be used interchangeably in the present text.
The expression “glycosaminoglycan HMW” or “HMW glycosaminoglycan” refers to the “crosslinked glycosaminoglycan HMW” component and the “non-crosslinked glycosaminoglycan HMW” component if present, in the aqueous gel.
The expression “hyaluronic acid HMW”, also called “HA HMW” or “HMW HA”, refers to the “crosslinked hyaluronic acid HMW” (also called “crosslinked HA HMW” or “crosslinked HMW HA”) component and if applicable the “non-crosslinked hyaluronic acid HMW” (also called “non-crosslinked HA HMW” or “non-crosslinked HMW HA”) component if present, in the aqueous gel.
The “crosslinking rate” applied during the preparation of a “crosslinked glycosaminoglycan”, in particular a “crosslinked hyaluronic acid” corresponds to the ratio between the mass (or weight) of crosslinking agent and the mass of glycosaminoglycan, in particular of hyaluronic acid used for the preparation of said crosslinked glycosaminoglycan, in particular of said crosslinked hyaluronic acid. It is expressed as a percentage.
A crosslinked glycosaminoglycan and a composition comprising such a crosslinked glycosaminoglycan, in particular a crosslinked hyaluronic acid and a composition comprising such a crosslinked hyaluronic acid, can be qualified by their “modification degree” (MOD). This degree of modification corresponds to the molar amount of modifying agent linked to said glycosaminoglycan by one or more of its ends, expressed per 100 moles of disaccharide repeating units of glycosaminoglycan within the composition), in particular 100 moles of disaccharide repeating units of hyaluronic acid within the composition. The reference method for determining this degree of modification is Nuclear Magnetic Resonance spectroscopy (¹H NMR). Said modification degree is determined by the molar ratio of the crosslinking agent signals overs the glycosaminoglycan disaccharide units signal (glycosaminoglycan having been involved in the crosslinking or not).
A “crosslinked high-molar-mass (HMW) glycosaminoglycan” or “crosslinked high-molar-mass (HMW) glycosaminoglycan” refers to a crosslinked glycosaminoglycan prepared from a non-crosslinked high-molar-mass glycosaminoglycan. Preferentially, a crosslinked HMW glycosaminoglycan has a crosslinking rate of less than or equal to 8%, preferably less than or equal to 5%, and more preferentially less than 2%.
A “crosslinked high-molar-mass (HMW) hyaluronic acid” or “crosslinked high-molar-mass (HMW) hyaluronic acid” refers to a crosslinked hyaluronic acid prepared from a non-crosslinked high-molar-mass hyaluronic acid. Preferentially, a crosslinked HMW hyaluronic acid has a crosslinking rate of less than or equal to 8%, preferably less than or equal to 5%, and more preferentially less than 2%.
“The biomechanical and biological properties of glycosaminoglycans on skin physiology” include hydration and fibroblast activation in particular. For example, due to its biomechanical and biological properties, hyaluronic acid is involved in skin restructuring, regeneration and/or rejuvenation.
The “supplementation” of a composition represents the addition to this composition of at least one non-crosslinked low-molar-mass glycosaminoglycan. Preferentially, supplementation corresponds to the addition to a composition of at least one non-crosslinked low-molar-mass hyaluronic acid.
“Total glycosaminoglycan concentration” means the sum of the concentration of crosslinked high-molar-mass glycosaminoglycan and optionally non-crosslinked high-molar-mass glycosaminoglycan and the concentration of the glycosaminoglycan used for supplementation. This concentration is expressed in milligrams of glycosaminoglycan per gram of gel.
“Total hyaluronic acid concentration” means the sum of the concentration of crosslinked high-molar-mass hyaluronic acid and optionally non-crosslinked high-molar-mass hyaluronic acid and the concentration of the hyaluronic acid used for supplementation. This concentration is expressed in milligrams of glycosaminoglycan per gram of gel
A solution is considered “homogeneous” when its color, visual appearance and viscosity are perceived, by the naked eye and by touch, as uniform.
“Aqueous medium” means any liquid medium containing at least water as a solvent and which has the property of dissolving a glycosaminoglycan, in particular hyaluronic acid.
“Crosslinking agent” means any compound capable of introducing crosslinking between different glycosaminoglycan chains. As crosslinking agents suitable for the implementation of the invention, mention may be made in particular of bi- or multi-functional epoxy or non-epoxy crosslinking agents. Among the epoxy agents may be mentioned butanediol diglycidyl ether (BDDE), diepoxy octane, 1,2-bis(2,3-epoxypropyl)-2,3-ethylene, 1,2-bis(2,3-epoxypropyl)-2,3-emylene and mixtures thereof. Among the non-epoxy agents may be mentioned endogenous polyamines such as spermine, spermidine and putrescine, aldehydes, carbodiimides and divinylsulfone.
an aqueous gel» means both an aqueous gel obtainable by the method according to the invention or an aqueous gel according to the present invention when nothing else is specified.
“Soft gel” means a gel of which the viscoelastic distribution remains moderately elastic, that is to say, sufficiently viscous with a phase angle (δ) between 15° and 50° and a low elastic modulus G′, in other words a G′ less than or equal to 150 Pa preferably between 20 and 150 Pa, measured for a stress of 5 Pa at room temperature using a rheometer (TA Instruments DHR2) having a cone/plate geometry (cone angle 1°/plate diameter 40 mm) applying an oscillation stress sweep at a frequency of 1 Hz and an extrusion force less than or equal to 18 N, preferably less than 15 N, measured at a fixed speed of about 12.5 mm/min, in syringes with an internal diameter greater than or equal to 6.3 mm, with a needle of external diameter less than or equal to 0.3 mm (30 G) and length ½”, at room temperature.
“Easy to inject” means a gel with an average extrusion force of less than 18 N, preferably less than 15 N, when measured with a dynamometer, at a fixed speed of about 12.5 mm/min, in syringes with an outer diameter greater than or equal to 6.3 mm, with a needle of outer diameter less than or equal to 0.3 mm (30 G) and length ½”, at room temperature.
A composition or gel “easily diffused in the tissues” means a composition for cutaneous applications of regeneration and improvement of the appearance of the skin which does not promote the formation of papules after its injection in the superficial zones of the skin, i.e., in the upper and middle dermis.
“In vivo durability” means the ability of the gel to remain at the treated site over time after its injection. An in vivo durable gel is a gel capable of remaining at the treated site for at least 1 month, preferentially at least 3 months, more preferentially at least 6 months, i.e., capable of maintaining after application an overall aesthetic improvement for at least 1 month, preferentially at least 3 months, more preferentially at least 6 months, in other words capable of maintaining after application at least a grade 1 or 2 improvement on a subjective scale of evaluation of the overall aesthetic improvement comprising 5 grades: 1. Very improved; 2. Improved; 3. Unchanged; 4. Degraded; 5. Very degraded, and this for at least 1 month, preferentially at least 3 months, more preferentially at least 6 months.
The term “stability” characterizes the ability of a composition to maintain physicochemical properties (color, pH, homogeneity, turbidity, rheology, etc.) in accordance with its specifications, during storage of at least one year. The stability of a composition is generally monitored by observing and/or measuring one or more of its physicochemical parameters, in particular, one or more of its rheological parameters, such as its elastic modulus (G′) or viscosity (η).
“Sterile gel” means a gel that is safe for administration in or through the superficial areas of the skin, i.e., the upper and middle dermis. In particular, it is essential that the gel to be administered by an injection technique is free of any contaminant that could initiate an undesirable secondary reaction in the host organism.
The terms “additive”, “additional component” and “excipient” are used interchangeably and refer to any component compatible with application in and/or on the skin. The amount of additive used depends on the nature of said component, the desired effect, and the use of the composition according to the invention. This additive can be one of the following components or one of its derivatives: an anesthetic agent, an antioxidant, an amino acid, a vitamin, minerals, a nucleic acid, a nucleotide, a nucleoside, a co-enzyme, an adrenergic derivative, sodium dihydrogen phosphate monohydrate and/or dihydrate or sodium chloride.
As “anesthetic agent” mention may be made of Ambucaine, Amoxecaine, Amylein, Aprindine, Aptocaine, Articaine, Benzocaine, Betoxycaine, Bupivacaine, Butacaine, Butamben, Butanilicaine, Chlorobutanol, Chloroprocaine, Cinchocaine, Clodacaine, Cocaine, Cryofluorane, Cyclomethycaine, Dexivacaine, Diamocaine, Diperodon, Dyclonine, Etidocaine, Euprocine, Febuerine, Fomocaine, Guafecainol, Heptacaine, Hexylcaine, Hydroxyprocaine, Hydroxytetracaine, Isobutamben, Leucinocaine, Levobupivacaine. Levoxadrol. Lidamidine, Lidocaine, Lotucaine, Menglytate, Mepivacaine, Meprylcaine, Myrtecaine, Octacaine, Octodrine, Oxetacaine, Oxybuprocaine, Parethoxycaine, Paridocaine, Phenacaine, Piperocaine, Piridocaine, Polidocanol, Pramocaine, Prilocaine, Procaine, Propanocaine, Propipocaine, Propoxycaine, Proxymetacaine, Pyrrocaine, Quatacaine, Quinisocaine. Risocaine, Rodocaine, Ropivacaine, Tetracaine, Tolycaine, Trimecaine, and salts thereof.
As “antioxidant”, mention may be made of glutathione, ellagic acid, spermine, resveratrol, retinol, L-carnitine, polyols, polyphenols, flavonols, theaflavins, catechins, caffeine, ubiquinol, ubiquinone, alpha-lipoic acid.
As “amino acids” mention may be made of arginine, isoleucine, leucine, lysine, glycine, valine, threonine, proline, methionine, histidine, phenylalanine, tryptophan, cysteine.
As “vitamins”, mention may be made of vitamins E, A, C, B, and in particular vitamins B4, B5, B6, B8, B9, B7, B12 and, preferentially, pyridoxine.
As “minerals”, mention may be made of the salts of zinc, magnesium, calcium, potassium, manganese, sodium, copper.
As “co-enzymes”, mention may be made of the coenzymes Q10, CoA, NAD, NADP.
As “adrenergic derivative” mention may be made of adrenaline and noradrenaline.

Finally, it is understood that all numerical values indicated are not to be considered strictly but approximately according to the general convention, i.e., the last digit indicated of the numerical values corresponds to the precision of the measurement. For example, for a molar mass of 0.04 MDa, the margin of error is 0.035-0.044 MDa.

### Process According to the Invention

As specified above, the present text describes a process for preparing an aqueous gel requiring, on the one hand, an aqueous solution comprising at least one crosslinked glycosaminoglycan, also called “crosslinked glycosaminoglycan HMW” because it is formed from non-crosslinked glycosaminoglycan of high-molar-mass and a crosslinking agent, and on the other hand, an aqueous solution comprising at least one non-crosslinked glycosaminoglycan of low-molar-mass, called “non-crosslinked glycosaminoglycan LMW”, and to form a homogeneous mixture of all or part of said solutions.

The glycosaminoglycans used in a process according to the invention may be selected from hyaluronic acid, heparosan, chondroitin sulfate, dermatan sulfate, keratan sulfate and mixtures thereof.

The glycosaminoglycans used in the process of the invention may be of the same or different chemical nature.

In a particular embodiment, the glycosaminoglycans used in the process of the invention are of identical chemical nature and preferably are all hyaluronic acid.

More particularly, the present invention relates to a process for preparing an aqueous gel requiring, on the one hand, an aqueous solution comprising at least one crosslinked hyaluronic acid, also called “crosslinked HA HMW” because it is formed from non-crosslinked hyaluronic acid of high-molar-mass and a crosslinking agent, and on the other hand, an aqueous solution comprising at least one non-crosslinked hyaluronic acid of low-molar-mass, called “non-crosslinked HA LMW”, and to form a homogeneous mixture of all or part of said solutions, characterized in that it comprises the use of hyaluronic acid in a mass ratio of “non-crosslinked HA LMW” to “HA HMW” of 1:3 to 2:1.

In a particular embodiment, the hyaluronic acids used in the process of the invention are not chemically modified.

As previously indicated, the process according to the invention uses these glycosaminoglycans and preferably these hyaluronic acids, in the form of aqueous solutions particularly as defined in steps a) and b) of the process according to the invention.

These solutions of glycosaminoglycans, in particular of hyaluronic acids, are advantageously homogeneous.

They can be prepared according to a conventional method, for example by solubilization and/or dilution of a glycosaminoglycan, in particular of a hyaluronic acid, crosslinked or not crosslinked in an aqueous medium, such as phosphate-buffered saline. These preparations are advantageously carried out at room temperature, preferably at a temperature of between 15° C. and 25° C., under mechanical or manual stirring.

### Step A)

As regards the aqueous solution of “crosslinked glycosaminoglycan HMW” and in particular of “crosslinked HA HMW” considered in step a), they are first obtained by crosslinking at least one non-crosslinked glycosaminoglycan of mass-average molar mass greater than or equal to 0.50 MDa, preferentially greater than 1.00 MDa, more preferentially from 1.00 MDa to 4.00 MDa with at least one crosslinking agent; and in particular they are first obtained by crosslinking at least one non-crosslinked hyaluronic acid of mass-average molar mass greater than or equal to 0.50 MDa, preferentially greater than 1.00 MDa, more preferentially from 1.00 MDa to 4.00 MDa with at least one crosslinking agent.

The realization of such a crosslinking is clearly within the competence of the skilled person. Advantageously, the crosslinking agent is introduced in a crosslinking rate of less than or equal to 8% by mass, preferentially less than or equal to 5%, more preferentially less than 2%. Advantageously, the crosslinking agent is introduced in a molar ratio with respect to the total number of moles of glycosaminoglycan units, in particular with respect to the total number of moles of hyaluronic acid units, of from 0.001 to 0.25.

Thus, according to a preferred embodiment, said “crosslinked HA HMW” is prepared with a crosslinking ratio of less than or equal to 8% by mass, preferentially less than or equal to 5%, and more preferentially less than 2%.

This crosslinking is carried out at various temperatures and times to obtain the expected modification degree.

Thus, a crosslinking carried out at room temperature, i.e., a temperature varying from 15° C. to 25° C., may require a reaction time of more than 5 h, or even 10 h or more.

On the other hand, crosslinking stimulated by a stimulating element such as heating, UV exposure, microwave exposure or a catalyst, preferably through the use of heating, can have a significantly increased crosslinking degree and a duration of less than 5 hours.

In a particular embodiment, the crosslinking of uncrosslinked glycosaminoglycan, in particular of uncrosslinked hyaluronic acid of molar mass greater than or equal to 0.50 MDa, preferentially greater than 1.00 MDa, more preferentially ranging from 1.00 MDa to 4.00 MDa, is carried out at a temperature greater than 40° C., preferably greater than 50° C., more particularly ranging from 45° C. to 60° C. or, better still, from 50° C. to 55° C.

Such crosslinking has advantageously a duration of 30 to 300 minutes, preferentially of 100 to 240 minutes.

In a particularly preferred embodiment, the crosslinking of uncrosslinked hyaluronic acid of molar mass greater than or equal to 0.50 MDa, preferentially greater than 1.00 MDa, more preferentially ranging from 1.00 MDa to 4.00 MDa, is carried out at room temperature, i.e., a temperature ranging from 15° C. to 25° C.

Such a crosslinking has advantageously a reaction time of more than 5 h, or even greater than 10 h, in particular between 10 h and 100 h.

Preferentially, the crosslinking agent used in a process according to the invention is an epoxy crosslinking agent, preferentially 1,4-butanediol diglycidyl ether (BDDE).

In a particular embodiment where the crosslinking agent used is a bi- or multi-functional epoxy crosslinking agent, such as BDDE, the pH of the aqueous medium is basified or acidified in order to activate the crosslinking reaction. According to a particular embodiment, the aqueous medium is basified with generally a solution containing sodium hydroxide and preferably, the pH of the aqueous medium is greater than or equal to 11, more preferably the pH of the aqueous medium is greater than or equal to 12.

According to a preferred embodiment, the concentration of “crosslinked HMW glycosaminoglycan”, more particularly of “crosslinked HMW HA” of the aqueous medium of step a) is adjusted to form at the end of step c) an aqueous gel of which the concentration of “crosslinked HMW glycosaminoglycan”, more particularly of “crosslinked HMW HA”, is at least 5 mg/g with respect to the total mass of said aqueous gel.

Preferably, the process according to the invention uses an aqueous solution comprising 5 to 15 mg/g of “crosslinked HA HMW”.

### Step B)

Step b) of the process of the invention requires, for its part, to have an aqueous solution comprising at least one non-crosslinked glycosaminoglycan LMW”, in particular at least one “non-crosslinked HA LMW”.

Preferentially, the concentration of “non-crosslinked glycosaminoglycan LMW”, in particular of “non-crosslinked HA LMW” considered in the aqueous solution of step b) is adjusted to form at the end of step c) an aqueous gel of which the “non-crosslinked glycosaminoglycan LMW” concentration, in particular the “non-crosslinked HA LMW” concentration, is at least 3 mg/g, preferably 10 mg/g, with respect to the total mass of said aqueous gel.

Preferably, the process according to the invention uses an aqueous solution comprising from 50 to 60 mg / g of “uncrosslinked HA LMW”.

As detailed below, the process according to the invention may also comprise the use of a glycosaminoglycan, in particular a hyaluronic acid, distinct from those defined for steps a) and b).

### Step C)

As described above, step c) consists in forming a homogeneous mixture from at least some or all of the solutions obtained in steps a) and b).

The formation of a homogeneous mixture as considered in step c) advantageously involves at least one homogenization step.

The homogenization is advantageously carried out under gentle conditions in order to prevent a degradation of the glycosaminoglycan chains used.

This step c) of forming a homogeneous mixture of at least one crosslinked glycosaminoglycan and at least one non-crosslinked glycosaminoglycan, in particular at least one crosslinked hyaluronic acid and at least one non-crosslinked hyaluronic acid, can be carried out according to conventional homogenization methods, such as three-dimensional stirring, paddle mixer stirring, spatula stirring and/or a step of extrusion through at least one grid.

According to a preferential embodiment, step c) comprises at least one step of extruding the formed mixture through at least one grid. The adjustment of the characteristics of such a grid is part of the general knowledge of the skilled person. Classically, they are chosen as a function of the properties expected for the mixture to be formed and taking into account the respective specificities of the solutions of glycosaminoglycans, in particular of the solutions of hyaluronic acids, which are brought together.

According to a particular embodiment, this step c) thus comprises at least one step of homogenization by means of a paddle mixer, followed by at least one step of extruding the mixture through at least one grid.

Homogenization is considered satisfactory when the gel obtained has macroscopically, to the naked eye and to the touch, a homogeneous color, a uniform viscosity and is free of agglomerates. The duration of the homogenization is assessed by the person skilled in the art according to his general knowledge. Said homogenization may comprise several cycles, optionally spaced in time by a rest phase, in particular so as to assess the quality of homogenization of the glycosaminoglycans, in particular of the hyaluronic acids, within said gel.

More particularly, a step c) according to the present invention may comprise a homogenization with a total duration of less than 200 minutes, preferably less than 150 minutes, or even a duration ranging from 5 to 100 minutes.

### Non-Crosslinked HMW Glycosaminoglycan, in Particular Non-Crosslinked HMW Hyaluronic Acid

In a particular embodiment, a process according to the invention also considers the use of at least one non-crosslinked glycosaminoglycan of molar mass greater than or equal to 0.50 MDa, preferentially greater than 1.00 MDa, more preferentially ranging from 1.00 MDa to 4.00 MDa, called “non-crosslinked HMW glycosaminoglycan”, in particular the use of at least one non-crosslinked hyaluronic acid of mass-average molar mass greater than or equal to 0.50 MDa, preferentially greater than 1.00 MDa, more preferentially ranging from 1.00 MDa to 4.00 MDa, called “non-crosslinked HA HMW”.

Preferentially, said “non-crosslinked HMW glycosaminoglycan”, in particular said “non-crosslinked HA HMW”, is implemented within an aqueous solution. This aqueous solution may be distinct from or common to one of the aqueous solutions considered in steps a) and b).

According to an alternative embodiment, all or part of said “uncrosslinked glycosaminoglycan HMW”, in particular of said “uncrosslinked HA HMW”, is used in the aqueous solution of step b).

According to another alternative embodiment, all or part of said “non-crosslinked HMW glycosaminoglycan”, in particular said “non-crosslinked HA HMW”, is used in the form of an aqueous solution distinct from the aqueous solutions of steps a) and b). According to this alternative, the aqueous solution containing all or part of said “non-crosslinked HMW glycosaminoglycan”, in particular said “non-crosslinked HA HMW”, and the aqueous solution of step b) are mixed concomitantly or successively with the aqueous solution of step a).

Preferably, the process according to the invention uses, in step b), an aqueous solution comprising 5 to 15 mg / g of “uncrosslinked HA HMW”.

### Additives

In a particular embodiment, the present invention relates to a process as described above further comprising the implementation of at least one additive chosen from the group consisting of anesthetic agents, antioxidants, amino acids, vitamins, minerals, nucleic acids, nucleotides, nucleosides, co-enzymes, adrenergic derivatives, sodium dihydrogen phosphate monohydrate and / or dihydrate and sodium chloride.

### Sterilization

Thus, according to one aspect of the present invention, in order to maintain the sterility of said gel, the raw materials used are sterile and the preparation of said gel including its packaging in its administration device is carried out under controlled atmospheric conditions.

According to another aspect, the present invention relates to a method for preparing a sterile and injectable aqueous gel comprising at least one step consisting in implementing an aqueous gel obtained by a method according to the invention or an aqueous gel as defined in the present invention and a sterilization step, in particular in an autoclave.

More particularly, a process for preparing an aqueous gel according to the invention comprises at least the steps consisting in:
a) having an aqueous solution comprising at least one crosslinked glycosaminoglycan called “crosslinked glycosaminoglycan HMW” formed from non-crosslinked glycosaminoglycan of molar mass greater than or equal to 0.50 MDa, preferentially greater than 1.00 MDa, more preferentially ranging from 1.00 MDa to 4.00 MDa, and from a crosslinking agent
b) having an aqueous solution comprising at least one non-crosslinked glycosaminoglycan of molar mass ranging from 0.04 MDa to 0.30 MDa, preferentially from 0.08 MDa to 0.20 MDa, more preferentially from 0.08 MDa to 0.15 MDa, still more preferentially from 0.08 MDa to 0.10 MDa: called “non-crosslinked glycosaminoglycan LMW”,
c) forming a homogeneous mixture of all or part of said solutions from steps a) and b), which is sterilized, in particular in an autoclave.

More particularly, a process for preparing an aqueous gel according to the invention comprises at least the steps consisting in:
a) having an aqueous solution comprising at least one crosslinked hyaluronic acid, called “crosslinked HA HMW”, formed from a non-crosslinked hyaluronic acid of mass-average molar mass greater than or equal to 0.50 MDa, preferentially greater than 1.00 MDa, more preferentially ranging from 1.00 MDa to 4.00 MDa, and a crosslinking agent;
b) having an aqueous solution comprising at least one non-crosslinked hyaluronic acid, called “non-crosslinked HA LMW”, of mass-average molar mass ranging from 0.04 MDa to 0.30 MDa, preferentially from 0.08 MDa to 0.20 MDa; more preferentially from 0.08 MDa to 0.15 MDa,
c) forming a homogeneous mixture of all or part of said solutions of steps a) and b) which is sterilized, in particular in an autoclave, characterized in that it comprises the use of hyaluronic acid in a mass ratio of “uncrosslinked HA LMW” to “IIA HMW” of 1:3 to 2:1.

This sterilization is preferably carried out on a product already packaged in its administration device, generally a syringe.

Thus, advantageously, an aqueous gel obtainable by a process according to the invention is packaged in a syringe prior to its sterilization.

Preferably, said method comprises a single sterilization step.

Preferably, said sterilization step is carried out by thermal means, for example in an autoclave, in particular at a temperature between 120° C. and 140° C.

In particular, the sterilization step is carried out in an autoclave, under moist heat conditions, at a plateau temperature greater than or equal to 121° C., preferably between 121° C. and 135° C. with an F0 greater than 15 (sterilizing value). According to the present invention, the sterilizing value F0 corresponds to the time required, in minutes, at 121° C., to inactivate 90% of the population of microorganisms present in the product to be sterilized.

### Aqueous Gel

As mentioned above, one aspect of the present invention is a hyaluronic acid-based aqueous gel comprising crosslinked hyaluronic acid, characterized in that said gel comprises at least:
a water-soluble hyaluronic acid with a mass-average molar mass ranging from 0.02 MDa to 0.30 MDa, called “sHA LMW”, in an amount of from 30% to 70% by mass with respect to the total mass of hyaluronic acid; and
optionally, a water-soluble hyaluronic acid with a mass-average molar mass greater than 0.30 MDa, called “sHA HMW”,
and characterized in that total water-soluble hyaluronic acids have a modification degree less than or equal to 5%, preferentially less than or equal to 3%, more preferentially less than or equal to 1%, respective percentages of soluble hyaluronic acids being determined by diluting the aqueous gel within a pH 7.2 solution of 150 nM sodium nitrate containing 0.02 % NaN₃ at 25° C. for 5 days and centrifugating it at 4400 rpm for 10 minutes and filtering at 0.45 mm such diluted gel to obtain a filtrate then analysing said filtrate with a size exclusion chromatography instrument equipped with a multiangle light scattering (MALS) detector and a refractive index (RI) detector with a refractive index increment (dn/dc) set at 0.165 mL/g and a liquid chromathography pumping station equipped with a dual set of size exclusion columns adapted for molecules with mass-average molar mass comprised from 0.001 Da to 20 MDa, with a pH 7.2 mobile phase of sodium nitrate at a flow rate of 0.3 mL/min.

In a particular embodiment, the aqueous gel according to the invention is a soft gel, i.e. a gel having a phase angle (δ) between 15° and 50° and an elastic modulus G′ less than or equal to 150 Pa, preferably between 20 and 150 Pa, measured for a stress of 5 Pa at room temperature using a rheometer (TA Instruments DHR2) having a cone/plate geometry (cone angle 1°/plate diameter 40 mm) applying an oscillation stress sweep at a frequency of 1 Hz and an extrusion force less than or equal to 18 N, preferably less than 15 N, measured at a fixed speed of about 12.5 mm/min, in syringes with an internal diameter greater than or equal to 6.3 mm, with a needle of external diameter less than or equal to 0.3 mm (30 G) and length ½”, at room temperature.

In a particular embodiment, the aqueous gel according to the invention comprises from 35% to 65% by mass of “sHA LMW” with respect to the total mass of said aqueous gel.

In a particular embodiment, in the aqueous gel according to the invention, said water-soluble hyaluronic acid called “sHA LMW” has a mass-average molar mass ranging from 0.04 MDa to 0.20 MDa, preferably from 0.04 MDa to 0.15 MDa.

In a particular embodiment, in the aqueous gel according to the invention, said water-soluble hyaluronic acid called “sHA HMW” has a mass-average molar mass greater than 0.30 MDa and lower than or equal to 4.00 MDa.

In a particular embodiment, the aqueous gel according to the invention comprises from 10% to 30% by mass, preferably from 15% to 20% by mass of “sHA HMW” with respect to the total mass of said aqueous gel.

In a particular embodiment, in the aqueous gel according to the invention, said crosslinked hyaluronic acid is present in an amount of at least 5 mg/g with respect to the total mass of said aqueous gel.

In a particular embodiment, in the aqueous gel according to the invention, said water-soluble hyaluronic acid called “sHA LMW” is present in an amount of at least 3 mg/g, preferentially 10 mg/g with respect to the total mass of said aqueous gel.

In a particular embodiment, in the aqueous gel according to the invention, the total hyaluronic acid concentration is from 10 to 40 mg/g, preferentially from 15 to 35 mg/g, more preferentially from 20 to 30 mg/g with respect to the total mass of said aqueous gel.

In a particular embodiment, in the aqueous gel according to the invention, the modification degree of the total hyaluronic acids is less than or equal to 5%, preferably less than or equal to 3%, more preferentially less than or equal to 1%.

According to a particular embodiment, in an aqueous gel as defined in the invention:
the crosslinked hyaluronic acid is present in an amount ranging from 6 mg/g to 12 mg/g with respect to the total mass of the aqueous gel,
the water-soluble hyaluronic acid called “sHA LMW” is present in an amount ranging from 12 mg/g to 20 mg/g with respect to the total mass of the aqueous gel,
the water-soluble hyaluronic acid called “sHA HMW” is present in an amount ranging from 3 mg/g to 6 mg/g with respect to the total mass of the aqueous gel,
said aqueous gel comprising a total hyaluronic acid concentration of 20 to 30 mg/g with respect to the total mass of the aqueous gel.

In a particular embodiment, an aqueous gel according to the invention is subjected to a heat sterilization step, for example in an autoclave.

Hyaluronic acid is known to be highly sensitive to strong acidic and alkali pH, high temperatures (e.g. during heat sterilization), oxidation (e.g. reactive oxygen species) and enzymatic activity (Stem R, Kogan G, Jedrzejas MJ, et al. The many ways to cleave hyaluronan. Biotechnol Adv 2007;25:537-57).

As a result, the manufacturing process notably the crosslinking conditions applied for producing compositions comprising crosslinked hyaluronic acid are prone to degrade hyaluronic acid chains releasing LMW soluble hyaluronic acid. Such LMW hyaluronic acid fragments are at least partially modified by the used crosslinking agent, such as by BDDE.

On the contrary, soluble hyaluronic acid from non-crosslinked LMW hyaluronic acid added during the preparation process are not modified with BDDE.

BDDE modification may be visualized by modification degree measurement (MOD). In this context, the higher the MOD of soluble hyaluronic acid, the higher the proportion of LMW hyaluronic acid fragments generated during preparation process of the studied gel and the lower the proportion of soluble hyaluronic acid from LMW hyaluronic acid added during the preparation process.

This is illustrated in Example 7.

The abovementioned hyaluronic acid cleavage explains that the molar mass of hyaluronic acid added within a gel before sterilization by heat is higher than the molar mass of soluble hyaluronic acid measured within a sterilized gel.

Such sensitivity is also true for other glycosaminoglycans.

Practically, “sHA” corresponds to:
uncrosslinked HA LMW added during the preparation process of the gel after the crosslinking step a); and.
when presents uncrosslinked HA HMW added during the preparation process of the gel after crosslinking step a); and,
HA fragments released during the preparation process of the gel said HA fragments being at least partially modified with a crosslinking agent, such as BDDE.

Thus, “sHA LMW” quantity is at least equal to the quantity of:
uncrosslinked HA LMW added during the preparation process of the gel after crosslinking step a); and,
when present, uncrosslinked HA HMW added during the preparation process of the gel after crosslinking step a).
On the contrary, “nsHA” corresponds to crosslinked HA.

According to a particular embodiment, an aqueous gel according to the invention comprises at least:
a water-soluble hyaluronic acid with a mass-average molar mass ranging from 0.04 MDa to 0.20 MDa, called “sHA LMW”, in an amount of from 30% to 70% by mass with respect to the total mass of hyaluronic acid; and

optionally, a water-soluble hyaluronic acid with a mass-average molar mass greater than 0.30 MDa and lower than or equal to 4.00 MDa, called “sHA HMW”,
and in that total water-soluble hyaluronic acids have a modification degree less than or equal to 5%, preferentially less than or equal to 3%, more preferentially less than or equal to 1%, respective percentages of soluble hyaluronic acids being determined by diluting the aqueous gel within a pH 7.2 solution of 150 nM sodium nitrate containing 0.02 % NaN₃ at 25° C. for 5 days and centrifugating it at 4400 rpm for 10 minutes and filtering at 0.45 mm such diluted gel to obtain a filtrate then analysing said filtrate with a size exclusion chromatography instrument equipped with a multiangle light scattering (MALS) detector and a refractive index (RI) detector with a refractive index increment (dn/dc) set at 0.165 mL/g and a liquid chromathography pumping station equipped with a dual set of size exclusion columns adapted for molecules with mass-average molar mass comprised from 0.001 Da to 20 MDa, with a pH 7.2 mobile phase of sodium nitrate at a flow rate of 0.3 mL/min. According to a particular embodiment, an aqueous gel according to the invention comprises at least:
a water-soluble hyaluronic acid with a mass-average molar mass ranging from 0.04 MDa to 0.15 MDa, called “sHA LMW”, in an amount of from 30% to 70% by mass with respect to the total mass of hyaluronic acid; and
optionally, a water-soluble hyaluronic acid with a mass-average molar mass greater than 0.30 MDa and lower than or equal to 4.00 MDa, called “sHA HMW”,
and in that total water-soluble hyaluronic acids have a modification degree less than or equal to 5%, preferentially less than or equal to 3%, more preferentially less than or equal to 1%, respective percentages of soluble hyaluronic acids being determined by diluting the aqueous gel within a pH 7.2 solution of 150 nM sodium nitrate containing 0.02 % NaN₃ at 25° C. for 5 days and centrifugating it at 4400 rpm for 10 minutes and filtering at 0.45 mm such diluted gel to obtain a filtrate then analysing said filtrate with a size exclusion chromatography instrument equipped with a multiangle light scattering (MALS) detector and a refractive index (RI) detector with a refractive index increment (dn/dc) set at 0.165 mL/g and a liquid chromathography pumping station equipped with a dual set of size exclusion columns adapted for molecules with mass-average molar mass comprised from 0.001 Da to 20 MDa, with a pH 7.2 mobile phase of sodium nitrate at a flow rate of 0.3 mL/min.

According to a particular embodiment, an aqueous gel according to the invention comprises at least:
a water-soluble hyaluronic acid with a mass-average molar mass ranging from 0.02 MDa to 0.30 MDa, called “sHA LMW”, in an amount of from 35% to 65% by mass with respect to the total mass of hyaluronic acid; and
optionally, a water-soluble hyaluronic acid with a mass-average molar mass greater than 0.30 MDa, called “sHA HMW”, and in that total water-soluble hyaluronic acids have a modification degree less than or equal to 5%, preferentially less than or equal to 3%, more preferentially less than or equal to 1%,
respective percentages of soluble hyaluronic acids being determined by diluting the aqueous gel within a pH 7.2 solution of 150 nM sodium nitrate containing 0.02 % NaN₃ at 25° C. for 5 days and centrifugating it at 4400 rpm for 10 minutes and filtering at 0.45 mm such diluted gel to obtain a filtrate then analysing said filtrate with a size exclusion chromatography instrument equipped with a multiangle light scattering (MALS) detector and a refractive index (RI) detector with a refractive index increment (dn/dc) set at 0.165 mL/g and a liquid chromathography pumping station equipped with a dual set of size exclusion columns adapted for molecules with mass-average molar mass comprised from 0.001 Da to 20 MDa, with a pH 7.2 mobile phase of sodium nitrate at a flow rate of 0.3 mL/min.

According to a particular embodiment, an aqueous gel according to the invention comprises at least:
a water-soluble hyaluronic acid with a mass-average molar mass ranging from 0.04 MDa to 0.20 MDa, called “sHA LMW”, in an amount of from 35% to 65% by mass with respect to the total mass of hyaluronic acid; and

optionally, a water-soluble hyaluronic acid with a mass-average molar mass greater than 0.30 MDa and lower than or equal to 4.00 MDa, called “sHA HMW”,
and in that total water-soluble hyaluronic acids have a modification degree less than or equal to 5%, preferentially less than or equal to 3%, more preferentially less than or equal to 1%, respective percentages of soluble hyaluronic acids being determined by diluting the aqueous gel within a pH 7.2 solution of 150 nM sodium nitrate containing 0.02 % NaN₃ at 25° C. for 5 days and centrifugating it at 4400 rpm for 10 minutes and filtering at 0.45 mm such diluted gel to obtain a filtrate then analysing said filtrate with a size exclusion chromatography instrument equipped with a multiangle light scattering (MALS) detector and a refractive index (RI) detector with a refractive index increment (dn/dc) set at 0.165 mL/g and a liquid chromathography pumping station equipped with a dual set of size exclusion columns adapted for molecules with mass-average molar mass comprised from 0.001 Da to 20 MDa, with a pH 7.2 mobile phase of sodium nitrate at a flow rate of 0.3 mL/min.

According to a particular embodiment, an aqueous gel according to the invention comprises at least:
a water-soluble hyaluronic acid with a mass-average molar mass ranging from 0.04 MDa to 0.15 MDa, called “sHA LMW”, in an amount of from 35% to 65% by mass with respect to the total mass of hyaluronic acid; and
optionally, a water-soluble hyaluronic acid with a mass-average molar mass greater than 0.30 MDa and lower than or equal to 4.00 MDa, called “sHA HMW”, and in that total water-soluble hyaluronic acids have a modification degree less than or equal to 5%, preferentially less than or equal to 3%, more preferentially less than or equal to 1%,
respective percentages of soluble hyaluronic acids being determined by diluting the aqueous gel within a pH 7.2 solution of 150 nM sodium nitrate containing 0.02 % NaN₃ at 25° C. for 5 days and centrifugating it at 4400 rpm for 10 minutes and filtering at 0.45 mm such diluted gel to obtain a filtrate then analysing said filtrate with a size exclusion chromatography instrument equipped with a multiangle light scattering (MALS) detector and a refractive index (RI) detector with a refractive index increment (dn/dc) set at 0.165 mL/g and a liquid chromathography pumping station equipped with a dual set of size exclusion columns adapted for molecules with mass-average molar mass comprised from 0.001 Da to 20 MDa, with a pH 7.2 mobile phase of sodium nitrate at a flow rate of 0.3 mL/min. As mentioned above, the present invention also relates to an aqueous gel obtainable by a process according to the invention.

At the end of step c), a homogeneous aqueous gel is obtained, containing at least one “non-crosslinked LMW glycosaminoglycan” and “HMW glycosaminoglycan” including at least one “crosslinked HMW glycosaminoglycan”.

Advantageously, an aqueous gel obtainable by a process in accordance with the invention comprises a total glycosaminoglycan concentration ranging from 10 to 40 mg/g, preferentially from 15 to 35 mg/g, more preferentially from 20 to 30 mg/g with respect to its total mass.

An aqueous gel obtainable by a process in accordance with the invention may also be characterized by a mass ratio of “uncrosslinked glycosaminoglycan LMW” to “glycosaminoglycan HMW” ranging from 1:4 to 4:1, preferentially from 1:3 to 5:3, more preferentially from 1:2 to 2:1.

According to a particular embodiment, said aqueous gel has a modification degree of said glycosaminoglycans of less than 5%, preferably less than 3%, more preferentially less than 1%. More particularly, at the end of step c), a homogeneous aqueous gel is obtained, containing at least one “uncrosslinked HA LMW” and “HA HMW” including at least one “crosslinked HA HMW”.

Advantageously, an aqueous gel obtainable by a process in accordance with the invention comprises a total hyaluronic acid concentration ranging from 10 to 40 mg/g, preferentially from 15 to 35 mg/g, more preferentially from 20 to 30 mg/g with respect to its total mass.

According an embodiment the aqueous gel obtainable by a process in accordance with the invention is also characterized by a mass ratio of “uncrosslinked HA LMW” to “HA HMW” ranging from 1:3 to 5:3, preferentially from 1:2 to 5:3.

According to a particular embodiment, said aqueous gel has a modification degree of said hyaluronic acids of less than or equal to 5%, preferably less than or equal to 3%, more preferentially less than or equal to 1%.

According to another particular embodiment, said aqueous gel obtained by a process according to the present invention is a soft gel that is to say a gel having a phase angle (δ) between 15 ° and 50 ° and an elastic modulus G′ less than or equal to 150 Pa, preferably between 20 and 150 Pa, measured for a stress of 5 Pa at room temperature using a rheometer (TA Instruments DHR2) having a cone/plate geometry (cone angle 1°/plate diameter 40 mm) applying an oscillation stress sweep at a frequency of 1 Hz and an extrusion force less than or equal to 18 N, preferably less than 15 N, measured at a fixed rate of about 12.5 mm/min, in syringes with an internal diameter greater than or equal to 6.3 mm, with a needle of external diameter less than or equal to 0.3 mm (30 G) and length ½”, at room temperature.

According to an embodiment of the invention, said aqueous gel is obtained at the end of a process implementing:
in step a), an aqueous solution comprising from 5 to 15 mg/g of “crosslinked glycosaminoglycan HMW”;
in step b), an aqueous solution comprising from 50 to 60 mg/g of “uncrosslinked glycosaminoglycan LMW” and from 5 to 20 mg/g of “uncrosslinked glycosaminoglycan HMW” and,
said gel formed has a total glycosaminoglycan concentration of 20 to 30 mg/g with respect to its total mass.

According to an embodiment of the invention, said aqueous gel is obtained at the end of a process implementing:
in step a), an aqueous solution comprising from 5 to 15 mg/g of “crosslinked HA HMW”;
in step b), an aqueous solution comprising from 50 to 60 mg/g of “uncrosslinked HA LMW” and from 5 to 20 mg/g of “uncrosslinked HA HMW” and in that,
said gel formed has a total hyaluronic acid concentration of 20 to 30 mg/g with respect to its total mass.

Representative of aqueous gels obtainable by a process in accordance with the invention and having a total glycosaminoglycan concentration of from 10 to 40 mg/g, preferentially from 15 to 35 mg/g, more preferentially from 20 to 30 mg/g with respect to its total mass, results from the implementation in particular of:
at least one crosslinked glycosaminoglycan formed from at least one non-crosslinked glycosaminoglycan of molar mass greater than or equal to 0.50 MDa, preferentially greater than 1.00 MDa, more preferentially ranging from 1.00 MDa to 4.00 MDa, called “crosslinked glycosaminoglycan HMW”;
at least one non-crosslinked glycosaminoglycan of molar mass of 0.04 MDa to 0.30 MDa, preferentially of 0.08 MDa to 0.20 MDa, more preferentially of 0.08 MDa to 0.15 MDa, still more preferentially of 0.08 MDa to 0.10 MDa, called “non-crosslinked glycosaminoglycan LMW”; and,
optionally, at least one non-crosslinked glycosaminoglycan of molar mass greater than or equal to 0.50 MDa, preferentially greater than 1 MDa, more preferentially ranging from 1.00 MDa to 4.00 MDa, called “non-crosslinked glycosaminoglycan HMW”.

Representative of aqueous gels obtainable by a process in accordance with the invention and having a total hyaluronic acid concentration of from 20 to 30 mg/g with respect to its total mass, results from the implementation in particular of:
in step a), an aqueous solution comprising from 5 to 15 mg/g of “crosslinked HA HMW”;
in step b), an aqueous solution comprising from 50 to 60 mg/g of “uncrosslinked HA LMW” and from 5 to 20 mg/g of “uncrosslinked HA HMW”.

Representative of aqueous gels obtainable by a process in accordance with the invention results from the implementation in particular of:
at least one crosslinked hyaluronic acid, called “crosslinked HA HMW”, formed from a non-crosslinked hyaluronic acid of mass-average molar mass greater than 1.00 MDa, and a crosslinking agent;
at least one non-crosslinked hyaluronic acid, called “non-crosslinked HA LMW”, of mass-average molar mass ranging from 0.08 MDa to 0.20 MDa; and
optionally, at least one non-crosslinked hyaluronic acid, called “non-crosslinked HA HMW”, of mass-average molar mass greater than 1.00 MDa,
the hyaluronic acid being used in a mass ratio of “uncrosslinked HA LMW” to “HA HMW” of 1:3 to 2:1.

Representative of aqueous gels obtainable by a process in accordance with the invention results from the implementation in particular of:
at least one crosslinked hyaluronic acid, called “crosslinked HA HMW”, formed from a non-crosslinked hyaluronic acid of mass-average molar mass ranging from 1.00 MDa to 4.00 MDa, and a crosslinking agent;
at least one non-crosslinked hyaluronic acid, called “non-crosslinked HA LMW”, of mass-average molar mass ranging from 0.08 MDa to 0.15 MDa; and
optionally, at least one non-crosslinked hyaluronic acid, called “non-crosslinked HA HMW”, of mass-average molar mass ranging from 1.00 MDa to 4.00 MDa,
the hyaluronic acid being used in a mass ratio of “uncrosslinked HA LMW” to “HA HMW”of 1:3 to 2:1.

Representative of aqueous gels obtainable by a process in accordance with the invention results from the implementation in particular of:
at least one crosslinked hyaluronic acid, called “crosslinked HA HMW”, formed from a non-crosslinked hyaluronic acid of mass-average molar mass greater than or equal to 0.50 MDa, and a crosslinkiiig agent;
at least one non-crosslinked hyaluronic acid, called “non-crosslinked HA LMW”, of mass-average molar mass ranging from 0.04 MDa to 0.30 MDa; and
optionally, at least one non-crosslinked hyaluronic acid, called “non-crosslinked HA HMW”, of mass-average molar mass greater than or equal to 0.50 MDa,
the hyaluronic acid being used in a mass ratio of “uncrosslinked HA LMW” to “HA HMW” of 1:3 to 5:3.

Representative of aqueous gels obtainable by a process in accordance with the invention results from the implementation in particular of:
at least one crosslinked hyaluronic acid, called “crosslinked HA HMW”, formed from a non-crosslinked hyaluronic acid of mass-average molar mass greater than 1.00 MDa, and a crosslinking agent;
at least one non-crosslinked hyaluronic acid, called “non-crosslinked HA LMW”, of mass-average molar mass ranging from 0.08 MDa to 0.20 MDa; and
optionally, at least one non-crosslinked hyaluronic acid, called “non-crosslinked HA HMW”, of mass-average molar mass greater than 1.00 MDa,
the hyaluronic acid being used in a mass ratio of “uncrosslinked HA LMW” to “HA HMW” of 1:3 to 5:3.

Representative of aqueous gels obtainable by a process in accordance with the invention results from the implementation in particular of:
at least one crosslinked hyaluronic acid, called “crosslinked HA HMW”, formed from a non-crosslinked hyaluronic acid of mass-average molar mass ranging from 1.00 MDa to 4.00 MDa, and a crosslinking agent;
at least one non-crosslinked hyaluronic acid, called “non-crosslinked HA LMW”, of mass-average molar mass ranging from 0.08 MDa to 0.15 MDa; and
optionally, at least one non-crosslinked hyaluronic acid, called “non-crosslinked HA HMW”, of mass-average molar mass ranging from 1.00 MDa to 4.00 MDa,
the hyaluronic acid being used in a mass ratio of “uncrosslinked HA LMW” to “HA HMW” of 1:3 to 5:3.

Representative of aqueous gels obtainable by a process in accordance with the invention results from the implementation in particular of:
at least one crosslinked hyaluronic acid, called “crosslinked HA HMW”, formed from a non-crosslinked hyaluronic acid of mass-average molar mass greater than or equal to 0.50 MDa, and a crosslinking agent;
at least one non-crosslinked hyaluronic acid, called “non-crosslinked HA LMW”, of mass-average molar mass ranging from 0.04 MDa to 0.30 MDa; and,
optionally, at least one non-crosslinked hyaluronic acid, called “non-crosslinked HA HMW”, of mass-average molar mass greater than or equal to 0.50 MDa,
the hyaluronic acid being used in a mass ratio of “uncrosslinked HA LMW” to “HA HMW” of 1:2 to 5:3.

Representative of aqueous gels obtainable by a process in accordance with the invention results from the implementation in particular of:
at least one crosslinked hyaluronic acid, called “crosslinked HA HMW”, formed from a non-crosslinked hyaluronic acid of mass-average molar mass greater than 1.00 MDa, and a crosslinking agent;
at least one non-crosslinked hyaluronic acid, called “non-crosslinked HA LMW”, of mass-average molar mass ranging from 0.08 MDa to 0.20 MDa; and,
optionally, at least one non-crosslinked hyaluronic acid, called “non-crosslinked HA HMW”, of mass-average molar mass greater than 1.00 MDa,
the hyaluronic acid being used in a mass ratio of “uncrosslinked HA LMW” to “HA HMW” of 1:2 to 5:3.

Representative of aqueous gels obtainable by a process in accordance with the invention results from the implementation in particular of:
at least one crosslinked hyaluronic acid, called “crosslinked HA HMW”, formed from a non-crosslinked hyaluronic acid of mass-average molar mass ranging from 1.00 MDa to 4.00 MDa, and a crosslinking agent;
at least one non-crosslinked hyaluronic acid, called “non-crosslinked HA LMW”, of mass-average molar mass ranging from 0.08 MDa to 0.15 MDa; and,
optionally, at least one non-crosslinked hyaluronic acid, called “non-crosslinked HA HMW”, of mass-average molar mass ranging from 1.00 MDa to 4.00 MDa,
the hyaluronic acid being used in a mass ratio of “uncrosslinked HA LMW” to “HA HMW” of 1:2 to 5:3.

Representative of aqueous gels obtainable by a process in accordance with the invention and having a total hyaluronic acid of from 10 to 40 mg/g, preferentially from 15 to 35 mg/g, more preferentially from 20 to 30 mg/g with respect to its total mass, results from the implementation in particular of:
at least one crosslinked hyaluronic acid formed from at least one non-crosslinked hyaluronic acid of mass-average molar mass greater than or equal to 0.50 MDa, preferentially greater than 1.00 MDa, more preferentially ranging from 1.00 MDa to 4.00 MDa, called “crosslinked HA HMW”, and a crosslinking agent;
at least one non-crosslinked hyaluronic acid of mass-average molar mass ranging from 0.04 MDa to 0.30 MDa, preferentially from 0.08 MDa to 0.20 MDa, more preferentially 0.08 MDa to 0.15 MDa, called “non-crosslinked HA LMW”; and,
optionally, at least one non-crosslinked hyaluronic acid of mass-average molar mass greater than or equal to 0.50 MDa, preferentially greater than 1.00 MDa, more preferentially ranging from 1.00 MDa to 4.00 MDa, called “non-crosslinked HA HMW”,
the hyaluronic acid being used in a mass ratio of “uncrosslinked HA LMW” to “HA HMW” of 1:3 to 2:1.

Representative of aqueous gels obtainable by a process in accordance with the invention and having a total hyaluronic acid of from 10 to 40 mg/g, with respect to its total mass, results from the implementation in particular of:
at least one crosslinked hyaluronic acid formed from at least one non-crosslinked hyaluronic acid of mass-average molar mass greater than 1.00 MDa, called “crosslinked HA HMW” and a crosslinking agent;
at least one non-crosslinked hyaluronic acid of mass-average molar mass ranging from 0.08 MDa to 0.20 MDa, called “non-crosslinked HA LMW”; and,
optionally, at least one non-crosslinked hyaluronic acid of mass-average molar mass greater than 1.00 MDa, called “non-crosslinked HA HMW”
the hyaluronic acid being used in a mass ratio of “uncrosslinked HA LMW” to “HA HMW” of 1:3 to 2:1.

Representative of aqueous gels obtainable by a process in accordance with the invention and having a total hyaluronic acid of from 20 to 30 mg/g, with respect to its total mass, results from the implementation in particular of:
at least one crosslinked hyaluronic acid formed from at least one non-crosslinked hyaluronic acid of mass-average molar mass greater than 1.00 MDa, called “crosslinked HA HMW” and a crosslinking agent;
at least one non-crosslinked hyaluronic acid of mass-average molar mass ranging from 0.08 MDa to 0.20 MDa, called “non-crosslinked HA LMW”; and,
optionally, at least one non-crosslinked hyaluronic acid of mass-average molar mass greater than 1.00 MDa, called “non-crosslinked HA HMW”,
the hyaluronic acid being used in a mass ratio of “uncrosslinked HA LMW” to “HA HMW” of 1:3 to 2:1.

Representative of aqueous gels obtainable by a process in accordance with the invention and having a total hyaluronic acid of from 10 to 40 mg/g, with respect to its total mass, results from the implementation in particular of:
at least one crosslinked hyaluronic acid formed from at least one non-crosslinked hyaluronic acid of mass-average molar mass of ranging from 1.00 MDa to 4.00 MDa, called “crosslinked HA HMW” and a crosslinking agent;
at least one non-crosslinked hyaluronic acid of mass-average molar mass ranging from 0.08 MDa to 0.15 MDa, called “non-crosslinked HA LMW”; and,
optionally, at least one non-crosslinked hyaluronic acid of mass-average molar mass of ranging from 1.00 MDa to 4.00 MDa, called “non-crosslinked HA HMW”,
the hyaluronic acid being used in a mass ratio of “uncrosslinked HA LMW” to “HA HMW” of 1:3 to 2:1.

Representative of aqueous gels obtainable by a process in accordance with the invention and having a total hyaluronic acid of from 20 to 30 mg/g, with respect to its total mass, results from the implementation in particular of:
at least one crosslinked hyaluronic acid formed from at least one non-crosslinked hyaluronic acid of mass-average molar mass of ranging from 1.00 MDa to 4.00 MDa, called “crosslinked HA HMW” and a crosslinking agent;
at least one non-crosslinked hyaluronic acid of mass-average molar mass ranging from 0.08 MDa to 0.15 MDa, called “non-crosslinked HA LMW”; and,
optionally, at least one non-crosslinked hyaluronic acid of mass-average molar mass of ranging from 1.00 MDa to 4.00 MDa, called “non-crosslinked HA HMW”,
the hyaluronic acid being used in a mass ratio of “uncrosslinked HA LMW” to “HA HMW” of 1:3 to 2:1.

Representative of aqueous gels obtainable by a process in accordance with the invention and having a total hyaluronic acid of from 10 to 40 mg/g, preferentially from 15 to 35 mg/g, more preferentially from 20 to 30 mg/g with respect to its total mass, results from the implementation in particular of:
at least one crosslinked hyaluronic acid formed from at least one non-crosslinked hyaluronic acid of mass-average molar mass greater than or equal to 0.50 MDa, preferentially greater than 1.00 MDa, more preferentially ranging from 1.00 MDa to 4.00 MDa, called “crosslinked HA HMW”, and a crosslinking agent;
at least one non-crosslinked hyaluronic acid of mass-average molar mass ranging from 0.04 MDa to 0.30 MDa, preferentially from 0.08 MDa to 0.20 MDa, more preferentially 0.08 MDa to 0.15 MDa, called “non-crosslinked HA LMW”; and,
optionally, at least one non-crosslinked hyaluronic acid of mass-average molar mass greater than or equal to 0.50 MDa, preferentially greater than 1.00 MDa, more preferentially ranging from 1.00 MDa to 4.00 MDa, called “non-crosslinked HA HMW”,
the hyaluronic acid being used in a mass ratio of “uncrosslinked HA LMW” to “HA HMW” of 1:3 to 5:3.

Representative of aqueous gels obtainable by a process in accordance with the invention and having a total hyaluronic acid of from 10 to 40 mg/g, preferentially from 15 to 35 mg/g, more preferentially from 20 to 30 mg/g with respect to its total mass, results from the implementation in particular of:
at least one crosslinked hyaluronic acid formed from at least one non-crosslinked hyaluronic acid of mass-average molar mass greater than or equal to 0.50 MDa, preferentially greater than 1.00 MDa, more preferentially ranging from 1.00 MDa to 4.00 MDa, called “crosslinked HA HMW”, and a crosslinking agent;
at least one non-crosslinked hyaluronic acid of mass-average molar mass ranging from 0.04 MDa to 0.30 MDa, preferentially from 0.08 MDa to 0.20 MDa, more preferentially 0.08 MDa to 0.15 MDa, called “non-crosslinked HA LMW”; and,
optionally, at least one non-crosslinked hyaluronic acid of mass-average molar mass greater than or equal to 0.50 MDa, preferentially greater than 1.00 MDa, more preferentially ranging from 1.00 MDa to 4.00 MDa, called “non-crosslinked HA HMW”,
the hyaluronic acid being used in a mass ratio of “uncrosslinked HA LMW” to “HA HMW” of 1:2 to 5:3.

Of course, an aqueous gel obtainable by a process in accordance with the invention may also comprise one or more additives conventionally considered in this type of formulation and in particular one or more additives as defined above.

Of course, the choice and the amount of additive(s) are adjusted so as not to raise any risk of incompatibility with the other compounds used in a gel according to the invention, in particular with said hyaluronic acids, and to be compatible with the uses as described below. These adjustments are within the general competence of the person skilled in the art.

In a particular embodiment, the present invention also relates to a composition comprising at least one aqueous gel as defined according to the invention, and if need be at least one additive selected from the group consisting of anesthetic agents, antioxidants, amino acids, vitamins, minerals, nucleic acids, nucleotides, nucleosides, co-enzymes, adrenergic derivatives, sodium dihydrogen phosphate monohydrate and/or dihydrate and sodium chloride.

In a preferred embodiment, said additive is an anesthetic agent as defined above and in particular lidocaine, mepivacaine or a salt thereof such as hydrochloride.

The gels obtained according to the process of the invention and the aqueous gels according to the invention are particularly useful as compositions for use in the prevention and/or treatment of alteration of the surface appearance of the skin.

The compositions according to the present invention can be cosmetic and/or dermatological compositions.

These compositions can be administered topically but also by injection in particular in the superficial zones of the skin, i.e., in the upper and middle dermis.

When an injection is considered it is of course necessary that the composition and/or aqueous gel is sterile and injectable.

When the composition according to the invention is a topical composition, it can be in any galenical form suitable for its application, for example, in the form of a cream, ointment, lotion, serum, gel or dispersion.

Any suitable excipients well-known by the skilled in the art, in an appropriate amount depending on the wished indication, can be comprised in a composition according to the invention.

According to yet another aspect, the present invention relates to an injectable aqueous gel sterilized according to a sterilization process as described above.

Representative of such an aqueous gel obtainable by a process in accordance with the invention are in particular those comprising:
0.6 to 0.8% by mass of at least one “crosslinked glycosaminoglycan HMW” with respect to the total mass of said aqueous gel;
1.4 to 1.8% by mass of at least one “non-crosslinked glycosaminoglycan LMW” with respect to the total mass of said aqueous gel; and,
0.2 to 0.4% by mass of at least one “non-crosslinked glycosaminoglycan HMW” with respect to the total mass of said aqueous gel;
and having a total glycosaminoglycan concentration between 20 and 30 mg/g, a phase angle (δ) between 15 ° and 50 ° and a G′ between 20 and 150 Pa measured for a stress of 5 Pa at room temperature using a rheometer (TA Instruments DHR2) having a cone/plate geometry (cone angle 1°/plate diameter 40 mm) applying an oscillation stress sweep at a frequency of 1 Hz and an extrusion force less than or equal to 15 N when measured with a dynamometer, at a fixed speed of about 12.5 mm/min, in syringes with an external diameter greater than or equal to 6.3 mm, with a needle with an external diameter of less than or equal to 0.3 mm (30 G) and with a length of ½”, at room temperature.

Representative of such an aqueous gel obtainable by a process in accordance with the invention are in particular those comprising
0.6 to 1.0% by mass of at least one “crosslinked HA HMW” with respect to the total mass of said aqueous gel;
1.4 to 1.8% by mass of at least one “non-crosslinked HA LMW” with respect to the total mass of said aqueous gel; and,
0.2 to 0.6% by mass of at least one “non-crosslinked HA HMW” with respect to the total mass of said aqueous gel;
and having a total hyaluronic acid concentration between 20 and 30 mg/g, a phase angle (δ) between 15 ° and 50 ° and a G′ between 20 and 150 Pa measured for a stress of 5 Pa at room temperature using a rheometer (TA Instruments DHR2) having a cone/plate geometry (cone angle 1°/plate diameter 40 mm) applying an oscillation stress sweep at a frequency of 1 Hz and an extrusion force less than or equal to 18 N when measured with a dynamometer, at a fixed speed of about 12.5 mm/min, in syringes with an external diameter greater than or equal to 6.3 mm, with a needle with an external diameter of less than or equal to 0.3 mm (30 G) and with a length of ½”, at room temperature.

### Kit

In particular, the present invention relates to a pre-filled syringe comprising an aqueous gel obtainable by the method according to the invention or an aqueous gel as defined in the present invention or a cosmetic and / or dermatological composition according to the invention.

According to one further aspect, the present invention relates to a kit comprising a pre-filled syringe comprising an aqueous gel obtainable by the method according to the invention or an aqueous gel as defined in the present invention or a cosmetic and / or dermatological composition according to the invention and instructions for use.

### Uses

According to another aspect, the present invention relates to an aqueous gel according to the invention or dermatological composition according to the invention for use in the prevention and/or treatment of an alteration of the surface appearance of the skin. Preferentially, the invention relates to an aqueous gel as defined in the invention or a dermatological composition as defined in the invention for use in the augmentation and/or filling of soft tissues.

Also described is an aqueous gel as defined in the present invention or a dermatological composition according to the invention for use in the prevention and/or treatment of altered viscoelastic or biomechanical properties of the skin.

Also described is an aqueous gel as defined in the present invention or a dermatological composition according to the invention for use in the prevention and/or treatment of cutaneous signs induced by external factors such as stress, air pollution, tobacco or prolonged exposure to ultraviolet (UV) light.

The present invention also relates to the non-therapeutic cosmetic use of an aqueous gel as defined in the invention or a cosmetic composition according to the invention to prevent and/or treat an alteration in the surface appearance of the skin, such as to prevent and/or treat cutaneous signs of chronological aging.

The present invention also relates to the non-therapeutic cosmetic use of an aqueous gel as defined in the invention or a cosmetic composition according to the invention for augmenting and/or filling soft tissues, in particular filling wrinkles.

The present invention also relates to a method for preventing and/or treating an alteration in the surface appearance of the skin comprising at least one step of topically applying on a subject such as a patient in need thereof or a step of administering by injection into the body of a subject such as a patient in need thereof an effective amount of an aqueous gel as defined in the invention or a dermatological composition according to the invention.

The present invention also relates to a method for augmenting and/or filling soft tissues, in particular for filling wrinkles, comprising at least one step of topically applying on a subject such as a patient in need thereof or a step of administering by injection into the body of a subject such as a patient in need thereof an effective amount of an aqueous gel as defined in the invention or a cosmetic and/or dermatological composition according to the invention.

### EXAMPLE

### Materials

Separate sets of samples were prepared for the different examples below. There are unavoidable variabilities between these series, notably due to the use of different batches of hyaluronic acid, or different sterilization cycles. Consequently, it is important to note that inter-example comparisons between these series are not appropriate.

### Methods

The following protocols are applied to study the viscoelastic properties of the compositions analyzed in the examples.

### Rheological Characterization of Samples

The viscoelastic properties of a composition are measured with a rheometer (TA Instruments DHR2) at room temperature with a cone/plate geometry (cone angle 1°/plate diameter 40 mm). An oscillation stress sweep measurement at a frequency of 1 Hz is performed. The elastic modulus (G′, in Pa), phase angle (δ, in °) and complex viscosity (η^{∗}) are measured for a stress of 5 Pa.

### Characterization of the Extrusion Forces of the Samples

Sample extrusion forces are determined using a dynamometer, at a fixed speed of 12.5 mm/min, a 25-mm drop path, in 1 mL COC syringes of 6.4 mm internal diameter, with a 30 G ½” needle, at room temperature.

### Characterization of Sample Degradation Resistance Over Time and Under Heat

Sterilization of hyaluronic acid injectable gels is generally carried out by autoclave (moist heat sterilization process).

The storage stability of these compositions can be evaluated in an accelerated manner, through forced degradation tests at high temperature (ASTM F1980-16).

Degradation of gels is characterized by a significant loss of elastic modulus (G′).

In this context, the percentage loss of G′ on sterilization (AG′ (%)) is a relevant indicator of the resistance of the gel to degradation, both over time and under heat sterilization.

For this, the change in G′ at sterilization is calculated as follows:

$$\begin{matrix}Δ {\text{G}}^{′} \left(\%\right) = \left[\left({\text{G}}^{′} \text{after sterilization} - {\text{G}}^{′} \text{before sterilization}\right) / {\text{G}}^{′}\right) \\ \left(\text{before sterilization}\right] \text{x 100}\end{matrix}$$

In addition, a percentage improvement in G′ is defined as follows:

$$\begin{matrix}{\text{G}}^{′} \text{improvement} \left(\%\right) = \left[\left(Δ {\text{G}}^{′} \text{reference sample} -\right)\right) \\ \left(\left(Δ {\text{G}}^{′} \text{studied sample}\right) / Δ {\text{G}}^{′} \text{reference sample}\right] \text{x 100}\end{matrix}$$

### Characterization of the Resistance of Samples to Enzymatic Degradation

To study the resistance of the samples to enzymatic degradation, 2 g of the tested gel is put in contact and homogenized with a hyaluronidase (HAase) solution in phosphate-buffered saline (0.5 U/mL of gel), then placed in an oven at 37° C. for 24 h.

Each sample is characterized by a rheology measurement before and after enzymatic treatment and the change in G′ before/after degradation is calculated as follows: ΔG′ = [(final G′ -initial G′) / initial G′] x 100. This change in G′ is a direct index of the degree of gel degradation in the presence of HAase.

### Size and Distribution Characterization of Water-soluble and Non Water-Soluble Hyaluronic Acid Present in Samples

The Mw and the quantity of sHA released from HA gels were determined by size exclusion chromatography (SEC) equipped with a multiangle laser light scattering detection (Dawn Neon MALS, Wyatt Technology Corp) and a refractive index detection (Optilab, Wyatt Technology Corp). The instrumentation used an Agilent Infinity LC system equipped with a dual set of size exclusion columns OHpak LB-806M held in series flow (Shodex) covering a wide range of mass-average molar mass (from 0.001 MDa to 20 MDa). A pH 7.2 mobile phase of 150 mM sodium nitrate (containing 0.02 % NaN₃) was used as eluent at a flow rate of 0.3 mL/min. Refractive index increment (dn/dc) was set at 0.165 mL/g in these conditions. Chromatograms were obtained and analyzed using ASTRA software (Wyatt Technology Corp).

The sample preparation consisted in diluting the aqueous gel within a pH 7.2 solution of 150 nM sodium nitrate containing 0.02 % NaN₃ at 25° C. for 5 days then centrifugating it at 4400 rpm for 10 minutes and filtering it at 0.45 mm to obtain a filtrate then analysing said filtrate with the SEC equipped as above mentioned.

The concentration of gel in the mobile phase for the dilution step as well as the injected volume in the SEC were adequately adjusted as a function of the supernatant composition to max out the signal-to-bruise ratio and avoid column overload.

sHA percentages that are determined correspond to the mass of water-soluble HA with respect to the total mass of HA within the sample.

### Modification Degree of Water-Soluble Hyaluronic Acid Present in Samples by ¹H NMR

3 g of each sample were incubated with 10 mL of water for injection for 5 days at room temperature. The samples were then slightly centrifugated (4400 rpm, 10 min) to separate the gel from the supernatant (containing the water-soluble HA) and filtered at 0.45 mm to remove insoluble gel aggregates. The supernatants were freeze-dried and dissolved with 1 mL of D20. The ¹H NMR analysis is performed on a 500 MHz Bruker Avance spectrometer). The degree of modification was determined by the molar ratio of the crosslinking agent signals overs the HA disaccharide units signal (HA being crosslinked or non-crosslinked).

### Example 1

### Effect of Supplementing a Crosslinked HMW Hyaluronic Acid Composition With Non-Crosslinked LMW Hyaluronic Acid

The samples studied are presented below.

Comparative sample 1-1 is a crosslinked hyaluronic acid gel sterilized in a syringe in an autoclave and prepared from HMW hyaluronic acid (4.00 MDa, HTL, Eur. Ph. pharmaceutical/medical grade) and BDDE (Sigma Aldrich, ≥ 95%) with a total hyaluronic acid concentration of 20 mg/g and a modification degree of 2%.

Sample 1-2, according to the invention, is a crosslinked hyaluronic acid gel sterilized in a syringe in an autoclave, with a total hyaluronic acid concentration of 20 mg/g and prepared from a 1:1 mixture of sample 1-1 before sterilization with a 20 mg/g solution of uncrosslinked LMW sodium hyaluronate (0.10 MDa, HTL, Eur. Ph. pharmaceutical/medical grade).

Comparative sample 1-3 is a crosslinked hyaluronic acid gel sterilized in a syringe in an autoclave, with a total hyaluronic acid concentration of 10 mg/g and prepared from a 1:1 mixture of sample 1-1 prior to sterilization with non-crosslinked hyaluronic acid-free phosphate-buffered saline solution.

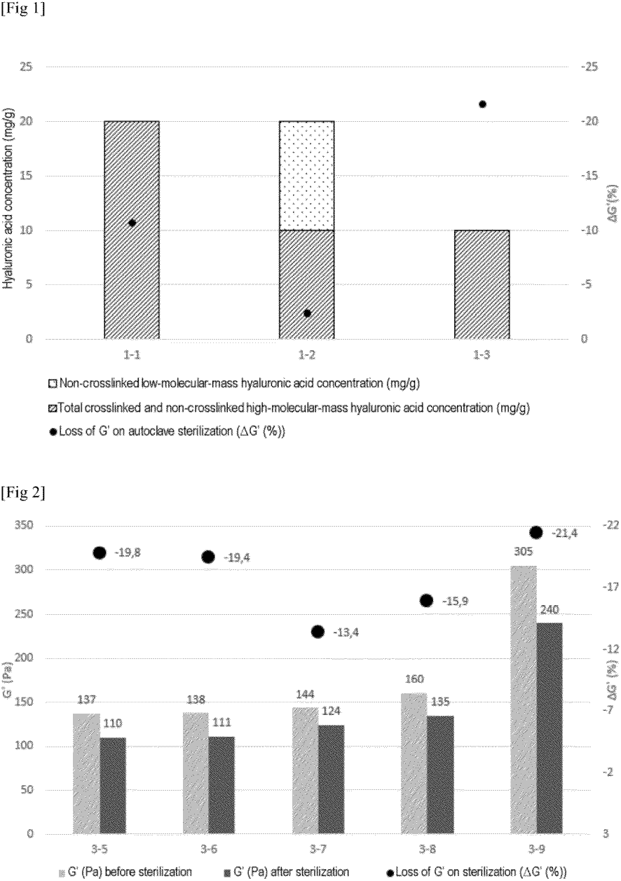

The characteristics of the samples, their G′ and their loss of G′ on sterilization are summarized in Table 1 below and in FIG. 1.

TABLE 1

| Samples | Crosslinked HMW HA concentration (mg/g) | Non-crosslinked LMW HA concentration (0.10 MDa) (mg/g) | Total HA concentration (mg/g) | ΔG′ (%) |
|---|---|---|---|---|
| 1-1 (reference) | 20 | 0 | 20 | -10.8 |
| 1-2 (invention) | 10 | 10 | 20 | -2.4 |
| 1-3 (comparative) | 10 | 0 | 10 | -21.6 |

The results show a better degradation resistance for compositions comprising LMW hyaluronic acid (sample 1-2 according to the invention) compared with a composition of the same total hyaluronic acid concentration but not comprising LMW hyaluronic acid (sample 1-1 (reference)).

Furthermore, it appears that the higher the concentration of crosslinked HMW hyaluronic acid, the greater the stability of the composition. Thus, sample 1-3 (comparative, 10 mg/g HMW hyaluronic acid) shows a greater loss of G′ (ΔG′= -21.6%) than sample 1-1 (comparative) comprising 20 mg/g HMW hyaluronic acid (ΔG′= -10.8%). Nevertheless, this increase in stability is much lower than that found for sample 1-2 (invention) comprising non-crosslinked LMW hyaluronic acid (ΔG′= -2.4%).

Consequently, it is not the simple increase in the total hyaluronic acid concentration that is associated with the increase in the degradation resistance of the compositions, but rather the supplementation of the relevant HMW hyaluronic acid composition.

### Example 2

### Effect of Supplementing a Composition Based on Crosslinked HMW Hyaluronic Acid, Optionally Comprising a Non-Crosslinked HMW Hyaluronic Acid, with a LMW Hyaluronic Acid

A sample 2-1 is prepared as follows.

Preparation of a BDDE crosslinked hyaluronic acid gel in basic medium from uncrosslinked HMW hyaluronic acid (HTL, 4.00 MDa, Eur. Ph. pharmaceutical/medical grade). The resulting crosslinked fraction is neutralized and swollen in phosphate-buffered saline to obtain a final gel with 15 mg/g NaHA. It is then dialyzed. At this stage, the gel has an elastic modulus G′ of about 80 Pa to 120 Pa and a phase angle δ of 7 ° to 10 °.

A 15 mg/g solution of uncrosslinked HMW sodium hyaluronate (4.00 MDa, HTL, Eur. Ph. pharmaceutical/medical grade) is added to the crosslinked fraction to reach a proportion of 30% by mass with respect to the total mass of hyaluronic acid in the composition. The gel is homogenized, extruded through a grid and filled into 1 mL COC syringes. Finally, the gel is autoclaved.

Sample 2-2 is prepared similarly to sample 2-1, but the composition of the uncrosslinked hyaluronic acid gel used has been modified so that the final gel further includes 10 mg/g of uncrosslinked LMW hyaluronic acid (0.10 MDa, HTL, Eur. Ph. pharmaceutical/medical grade).

Sample 2-3 is prepared as sample 2-1 but without non-crosslinked 4.00 MDa hyaluronic acid. Sample 2-4 is prepared as sample 2-1 but replacing the used non-crosslinked 4.00 MDa hyaluronic acid with 0.10 MDa hyaluronic acid.

For each of these samples, the variation (in percentage) of G′ is calculated. The results obtained are summarized in the tables below.

Table 2 shows the stabilization of a mixture of crosslinked and non-crosslinked hyaluronic acid.

TABLE 2

| Samples | HMW HA | Non-crosslinked LMW HA added (0.10 MDa) | ΔG′ (%) |
|---|---|---|---|
| 2-1 (comparative) | crosslinked + non-crosslinked | No | -25 |
| 2-2 (invention) | crosslinked + non-crosslinked | Yes | -15 |

Table 3 shows the stabilization of crosslinked hyaluronic acid alone.

TABLE 3

| Samples | HMW HA | Non-crosslinked LMW HA added (0.10 MDa) | ΔG′ (%) |
|---|---|---|---|
| 2-3 (comparative) | crosslinked alone | No | -15 |
| 2-4 (invention) | crosslinked alone | Yes | -10 |

The results show that the protective effect of non-crosslinked LMW hyaluronic acid during autoclave sterilization is observed both for compositions comprising crosslinked HMW hyaluronic acid alone (sample 2-4 vs. sample 2-3) and for compositions comprising a mixture of crosslinked and non-crosslinked HMW hyaluronic acid (sample 2-2 vs. sample 2-1).

Thus, the addition of non-crosslinked LMW hyaluronic acid to compositions comprising a crosslinked HMW hyaluronic acid alone (sample 2-4) or in mixture with a non-crosslinked HMW hyaluronic acid (sample 2-2) advantageously increases their resistance to degradation.

### Example 3

### Study of the Rheology and Stability of Hyaluronic Acid Gels Comprising Non-Crosslinked Hyaluronic Acid of Different Molar Masses

### Rheology of Hyaluronic Acid Solutions of Different Molar Mass

Solutions of 10 mg/g (1%) of non-crosslinked hyaluronic acid of varying molar masses (0.04 MDa, 0.10 MDa, 0.20 MDa and 1.50 MDa, HTL, Eur. Ph. pharmaceutical/medical grade) are prepared in phosphate-buffered saline. The elastic modulus (G′) and complex viscosity (η^{∗}) of each of these fractions are determined and detailed in Table 4 below.

TABLE 4

| Samples | Non-crosslinked hyaluronic acid (MDa) | η* (mPa.s) before sterilization | G′ (Pa) before sterilization |
|---|---|---|---|
| 3-1 | 0.04 | N.D.^{∗∗} | N.D.^{∗∗} |
| 3-2 | 0.10 | N.D.^{∗∗} | N.D.^{∗∗} |
| 3-3 | 0.20 | 21.2 | <1 |
| 3-4 | 1.50 | 2110 | 6.0 |

^{∗∗}N.D. Not determinable at 5 Pa stress (sample 3-1: η^{∗} = 2.55 mPa.s and G′ = 0.65 Pa to 0.6 Pa: sample 3-2: η^{∗} = 5.2 mPa.s and G′ = 0.0057 Pa to 1 Pa).

The above results show that a gel based on uncrosslinked hyaluronic acid of molar mass less than or equal to 0.20 MDa alone and concentrated at 1% has no significant elastic properties and very low viscosity.

### Mechanical Contribution

A mixture of crosslinked and non-crosslinked HMW hyaluronic acid, sample 3-5 (reference), is prepared as follows. Preparation of a BDDE-crosslinked hyaluronic acid gel in basic medium from uncrosslinked HMW hyaluronic acid (4.00 MDa, HTL, Eur. Ph. pharmaceutical/medical grade). The resulting crosslinked fraction is neutralized and swollen in phosphate-buffered saline to obtain a final gel with 15 mg/g NaHA. It is then dialyzed. At this stage, the gel has an elastic modulus G′ of about 80 to 120 Pa and a phase angle δ of 7 to 10 °. A 15 mg/g solution of uncrosslinked HMW sodium hyaluronate (4.00 MDa, Eur. Ph. pharmaceutical/medical grade) is added to the crosslinked fraction to reach a proportion of 30% by mass with respect to the total mass of hyaluronic acid in the composition. The gel is homogenized, extruded through a grid and filled into 1 mL COC syringes. Finally, the gel is autoclaved.

Samples 3-6 to 3-9 are prepared similarly to sample 3-5 but with a non-crosslinked hyaluronic acid gel further comprising an additional fixed concentration of 10 mg/g of non-crosslinked hyaluronic acid with respect to the total mass of the composition (1%).

or this addition, non-crosslinked hyaluronic acids of different molar masses are used, namely for samples 3-6 to 3-9, in order: 0.04 MDa, 0.10 MDa, 0.20 MDa and 1.50 MDa, HTL, Eur. Ph. pharmaceutical/medical grade).

The rheological characteristics of these samples are determined before and after sterilization, on 6 syringes of each sample. The results after sterilization are summarized in Table 5 below and in FIG. 2.

TABLE 5

| Samples | Non-crosslinked hyaluronic acid added (MDa) | δ (°)/ standard deviation (°) | G′ (Pa) before sterilization / standard deviation (Pa) | G′ (Pa) after sterilization / standard deviation (Pa) | F (N) / standard deviation (N) |
|---|---|---|---|---|---|
| 3-5 (reference) | - | 16.4 / 0.5 | 137 / 3 | 110 / 3 | 10.4. / 0.6 |
| 3-6 (invention) | 0.04 | 16.8 / 0.4 | 138 / 2 | 111 / 3 | 12.0 / 0.3 |
| 3-7 (invention) | 0.10 | 17.2 / 0.4 | 144 / 5 | 124 / 5^{∗} | 14.8 / 0.5 |
| 3-8 (invention) | 0.20 | 19.9 / 0.7 | 161 / 3 | 135 / 9^{∗} | 15.6 / 0.4 |
| 3-9 (comparative) | 1.50 | 27.6 / 4 | 305 / 3 | 240 / 21^{∗} | 16.0 / 0.7 |

^{∗}Mean significantly different from the mean of the Reference sample, p < 0.001, two-sample t test.

Thanks to a limited mechanical contribution of the uncrosslinked LMW hyaluronic acid, samples 3-6 to 3-8 remain soft gels, i.e., gels with a δ between 15° and 45°, a G′ less than or equal to 150 Pa and an extrusion force less than or equal to 18 N.

It is noted that the presence of additional LMW hyaluronic acid and thus an increased hyaluronic acid concentration in gels according to the invention does not affect their flexibility. On the other hand, the comparative sample 3-9, comprising an additional HMW hyaluronic acid, shows very significantly increased rheological characteristics and can no longer be considered as a soft gel (G′ > 150 Pa).

### Resistance to Degradation

The resistance to heat degradation of each sample is studied by calculating the percentage of G′ lost after undergoing the same autoclave cycle 1.

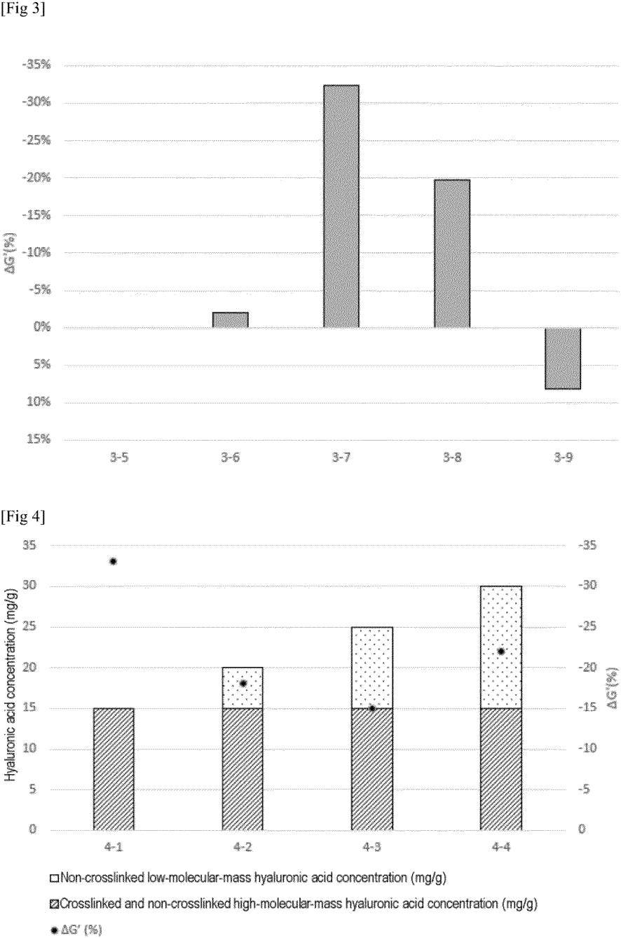

The results obtained are summarized in Table 6 below, in FIG. 2 and FIG. 3.

TABLE 6

| Samples | Non-crosslinked hyaluronic acid added (MDa) | ΔG′ (%) | G′ improvement (%) |
|---|---|---|---|
| 3-5 (reference) | - | -20 | 0 |
| 3-6 (invention) | 0.04 | -19 | 2 |
| 3-7 (invention) | 0.10 | -13 | 32 |
| 3-8 (invention) | 0.20 | -16 | 20 |
| 3-9 (comparative) | 1.50 | -21 | -8 |

As shown in FIG. 2, under the sterilization conditions implemented (identical for all samples tested), the reference sample 3-5 suffers a loss of about 20%.

The addition of a non-crosslinked HMW hyaluronic acid (1.50 MDa, sample 3-9) does not improve the loss of G′ on sterilization, which remains higher than that of the reference sample 3-5 (20%).

On the contrary, the supplementation according to the invention, i.e., the addition of a hyaluronic acid of molar mass ranging from 0.04 MDa to 0.20 MDa, more preferentially from 0.08 MDa to 0.10 MDa, is the source of a decrease in the loss of G′ on sterilization (comparison of samples 3-6 to 3-8 with sample 3-5), i.e., the source of an increase in the degradation resistance of the composition.

A composition supplemented with a LMW hyaluronic acid according to the invention, therefore, has a better degradation resistance compared with compositions with an identical total hyaluronic acid concentration, the same crosslinking rate of the crosslinked HMW hyaluronic acid fraction but no supplementation.

This improvement is minimal but already noticeable at the lowest molar mass (0.04 MDa) of hyaluronic acid used (sample 3-6). This improvement is significant with 0.10 MDa and 0.20 MDa hyaluronic acid (samples 3-7 and 3-8).

Thus, comparison of the means from the 6 measurements on 6 syringes of each product after sterilization shows that samples 3-7 and 3-8 have a significantly higher G′ value than sample 3-5 (FIGS. 2 and 3).

This increase in G′ is linked to both the interactions between the LMW HA and the HMW hyaluronic acid used in the formulation and especially to the effect of improved resistance to degradation associated with the presence of LMW HA in the composition (visible by the improvement in the values of loss of G′ on sterilization).

Thus, while the addition of a non-crosslinked HMW hyaluronic acid (for example 1.50 MDa) increases the mechanical properties of the composition without significantly improving its resistance to degradation, supplementation with a non-crosslinked LMW HA increases the resistance to degradation of a gel while maintaining the mechanical properties of a soft gel.

### Example 4

### Influence of Variation in Supplementation

For this example, the comparative sample 4-1 is identical to the sample 3-5 (comparative) prepared in Example 3. Different samples were prepared in order to study the effect of different concentrations of non-crosslinked LMW HA.

Sample 4-1 is compared with samples 4-2 to 4-4 prepared in a similar manner but for which the composition of the non-crosslinked hyaluronic acid gel used further comprises increasing concentrations of non-crosslinked LMW HA (0.10 MDa HTL, Eur. Ph. pharmaceutical-medical grade).

The proportion of non-crosslinked LMW HA (0.10 MDa, HTL, Eur. Ph. pharmaceutical/medical grade) corresponds to the ratio between the mass of non-crosslinked LMW hyaluronic acid and the mass of crosslinked HMW hyaluronic acid in the composition (%).

The rheological characteristics of the samples and their loss of G′ on sterilization ΔG′ (%) are summarized in Table 7 below and in FIG. 4.

TABLE 7

| Samples | Proportion of non-crosslinked LMW HA (%) | Non-crosslinked LMW HA concentration (mg/g) | Concentration of crosslinked and non-crosslinked HMW HA (mg/g) | δ (°) after sterilization | G′(Pa) after sterilization | ΔG′ (%) | F(N) |
|---|---|---|---|---|---|---|---|
| 4-1 (comparative) | 0 | 0 | 15 | 16.4 / 0.5 | 110 / 3 | -20 | 10.4 / 0.6 |
| 4-2 (invention) | 48 | 5 | 15 | 16.7 / 0.4 | 119 / 2 | -15 | 12.4 / 0.5 |
| 4-3 (invention) | 95 | 10 | 15 | 17.2 / 0.4 | 124 / 5 | -13 | 14.8 / 0.5 |
| 4-4 (invention) | 143 | 15 | 15 | 18.1 / 0.1 | 120 / 6 | -16 | 17.4 / 0.4 |

The stabilizing effect of supplementation is visible in all samples 4-2 to 4-4 according to the invention.

It is therefore possible to obtain the advantageous effect of supplementing gels comprising at least one crosslinked HMW hyaluronic acid with greater or lesser amounts of non-crosslinked LMW HA.

Moreover, the above results show the achievement of highly concentrated hyaluronic acid compositions (up to 30 mg/g total hyaluronic acid) that remain soft and easily injectable when implementing either low proportions of non-crosslinked LMW HA (about 50%) or higher proportions of non-crosslinked LMW HA (about 150%))

### Example 5

### Influence of the Variation of the HMW Hyaluronic Acid Concentration

For this example, the comparative sample 5-1 is prepared in the same manner as the sample 3-5 (comparative) prepared in Example 3. Samples 5-2 and 5-3 in accordance with the invention and comprising a lower concentration of HMW hyaluronic acid than in the previous examples were prepared for the purpose of studying the effect of a fixed concentration of non-crosslinked LMW hyaluronic acid on compositions of different concentrations of crosslinked and non-crosslinked HMW hyaluronic acids.

Sample 5-2 is prepared similarly to sample 5-1 but with a target concentration of 12 mg/g for the crosslinked hyaluronic acid fraction and 12 mg/g for the non-crosslinked HMW hyaluronic acid solution to which is added a sufficient amount of 0.10 MDa non-crosslinked hyaluronic acid (HTL. Eur. Ph. pharmaceutical/medical grade) to result in a concentration of 16 mg/g 0.10 MDa non-crosslinked hyaluronic acid in the final composition.

Sample 5-3 is also prepared similarly to the Reference sample but with a target concentration of 9 mg/g for the crosslinked HMW hyaluronic acid fraction and 9 mg/g for the non-crosslinked HMW hyaluronic acid solution to which is added a sufficient amount of 0.10 MDa non-crosslinked LMW hyaluronic acid (HTL, Eur. Ph. pharmaceutical/medical grade) to result in a concentration of 16 mg/g 0.10 MDa non-crosslinked hyaluronic acid in the final composition. The rheological characteristics of samples 5-1 to 5-3, their proportion of non-crosslinked LMW hyaluronic acid (%) and their loss of G′ on sterilization (ΔG′ (%)) are summarized in Table 8 below.

TABLE 8

| Samples | Proporti on of non-crosslink ed LMW HA (%) | Concentrati on of crosslinked and non-crosslinked HMW HA (mg/g) | Concentrati on of non-crosslinked LMW HA (mg/g) | G′ (Pa) after sterilizatio n | δ (◦) after steriliza tion | ΔG′ (%) | F(N) after sterilizatio n |
|---|---|---|---|---|---|---|---|
| 5-1 (comparative) | 0 | 15 | 0 | 98 ± 4 | 17.7 ± 0.5 | -25 | 11.9 ± 0.3 |
| 5-2 (invention) | 190 | 12 | 16 | 74 ±1 | 19.1 ± 0.6 | -19 | 18.2 ± 0.3 |
| 5-3 (invention) | 253 | 9 | 16 | 47 ± 8 | 21.3 ± 0.8 | -4 | 14.1 ± 0.9 |

Thanks to the addition of non-crosslinked LMW hyaluronic acid, it is possible to decrease the fraction of crosslinked and non-crosslinked HMW hyaluronic acid from 15 mg/g to 12 mg:g and observe only a slight decrease in G′ (sample 5-1 (comparative) vs. sample 5-2 (invention)). Moreover, thanks to the addition of non-crosslinked LMW hyaluronic acid, it is possible to go from a gel with 15 mg/g of hyaluronic acid to a gel with 28 mg/g of hyaluronic acid while maintaining the mechanical properties of a soft gel (sample 5-1 (comparative) vs. sample 5-2 (invention))

Sample 5-3 prepared with a very small amount of crosslinked and non-crosslinked HMW hyaluronic acid shows minimal loss of G′ on sterilization (only - 4%).

The presence of non-crosslinked LMW hyaluronic acid advantageously reduces the amount of crosslinked hyaluronic acid needed to prepare a soft gel. It also makes it possible to increase the total hyaluronic acid concentration. In other words, the supplementation thus makes it possible to obtain soft gels with a potentially increased biocompatibility (reduction of the proportion of crosslinked hyaluronic acid used) but also effective from a biomechanical and/or biological point of view.

### Example 6

### Resistance to Enzymatic Degradation

Sample 6-1 is prepared using the same protocol as sample 3-5 (comparative) prepared in Example 3.

Sample 6-2 is prepared in a similar manner, but the composition of the uncrosslinked hyaluronic acid gel used was modified so that the final gel further comprised 10 mg/g of uncrosslinked LMW hyaluronic acid (0.10 MDa, HTL. Eur. Ph. pharmaceutical/medical grade).

Sample 6-3 is also prepared similarly to sample 6-1, but the composition of the uncrosslinked hyaluronic acid gel used was modified so that the final gel further comprised 10 mg/g of uncrosslinked HMW hyaluronic acid (1.50 MDa, HTL, Eur. Ph. pharmaceutical/medical grade).

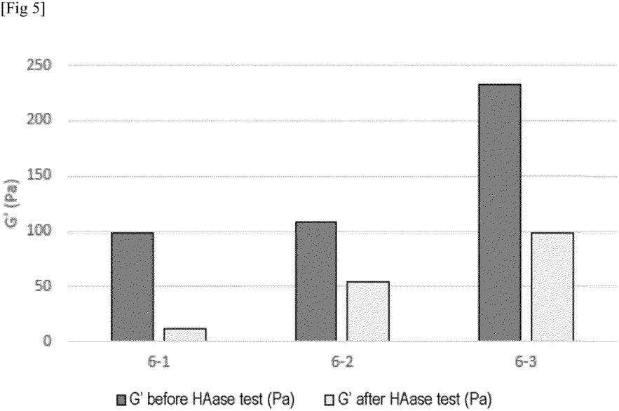

All these samples are submitted to an enzymatic degradation test with hyaluronidase (HAase). The rheological characteristics of the samples and their loss of G′ to enzymatic degradation are summarized in Table 9 below and in FIG. 5.

TABLE 9

| Samples | Concentration of crosslinked and non-crosslinked HMW HA (mg/g) | Concentration of non-crosslinked HA added (mg/g) | Non-crosslinked HA added (MDa) | G′ (Pa) before degradation / standard deviation | G′ (Pa) after degradation / standard deviation | ΔG′ (%) |
|---|---|---|---|---|---|---|
| 6-1 (comparative) | 15 | 0 | N/A | 110 / 3 | 89 / 1 | -19 |
| 6-2 (invention) | 15 | 10 | 0.10 | 124 / 5 | 110 / 3 | -12 |
| 6-3 (comparative) | 25 | 10 | 1.50 | 240 / 21 | 188 / 4 | -22 |

It is noted that samples 6-2 and 6-3 (comprising hyaluronic acids with molar masses of 0.10 MDa and 1.50 MDa, respectively) have very different starting properties. The properties of sample 6-3 are not desirable for skin regeneration and or skin improvement applications (see δ, G′ and F).

The loss of G′ to enzymatic degradation is lower in the presence of non-crosslinked LMW hyaluronic acid than without (sample 6-2 vs. sample 6-1) and even than in the presence of non-crosslinked HMW hyaluronic acid (sample 6-2 vs. sample 6-3). The increased resistance of the composition presented as complying with the invention (sample 6-2) is therefore not associated with the simple addition of non-crosslinked hyaluronic acid but specifically with the addition of non-crosslinked LMW hyaluronic acid.

The compositions supplemented according to the invention therefore have better resistance to enzymatic degradation compared with compositions having the same total hyaluronic acid concentration, the same crosslinking rate of the crosslinked hyaluronic acid fraction but comprising only HMW hyaluronic acid.

This increased resistance to degradation by hyaluronidase suggests the increased in vivo durability of the compositions according to the invention.

Finally, the above results highlight the protective effect of non-crosslinked LMW hyaluronic acid, thus the beneficial effect of supplementing with composition based on at least one crosslinked glycosaminoglycan.

### Example 7

### Characterization of Water Soluble and Non Water-soluble Hyaluronic Acid Present In Samples

Water-soluble and non water-soluble hyaluronic acid (sHA and nsHA) of several sterilized samples were characterized as described hereabove. The HA raw material Eur. Ph. pharmaceutical/medical grade are obtained from HTL.

The following compositions are considered references:
Sample 7-1: crosslinked HA HMW: a crosslinked HA gel prepared with HA 4,00 MDa alone within phosphate buffer; this crosslinked HA is prepared as the crosslinked HA part of sample 2-1 but is swollen to obtain a gel with a concentration of 10.5 mg/g of NaHA with respect to the total mass of the gel ;
Sample 7-2: non-crosslinked HA HMW: a non-crosslinked HA prepared with 4,00 MDa alone within phosphate buffer with a concentration of 4.5 mg/g of NaHA with respect to the total mass of the gel;
Sample 7-3: non-crosslinked HA LMW: a non-crosslinked HA prepared with HA 0.10 MDa alone within phosphate buffer with a concentration of 10 mg/g of NaHA with respect to the total mass of the gel.

The following three samples have variable proportions of HMW HA and LMW HA added during the process.
Sample 7-4: a gel, as previously presented sample 2-4, comprising, before sterilization, a crosslinked HA HMW prepared with HA 4.00 MDa alone and a non-crosslinked HA LMW prepared with HA 0.10 MDa ;
Sample 7-5: a gel comprising, before sterilization, a crosslinked HA prepared with HA HMW 4.00 MDa alone and a non-crosslinked HA LMW prepared with HA 0.10 MDa and a non-crosslinked HA HMW prepared with HA 4.00 MDa; said gel corresponds to sample 2-1 further comprising an additional fixed concentration of 10 mg/g of non-crosslinked HA LMW 0.10 MDa with respect to on the total mass of the gel (1%);
Sample 7-6: a gel, as previously presented sample 2-1, comprising crosslinked HA HMW prepared with HA 4.00 MDa and uncrosslinked HA HMW 4.00 MDa.

All samples 7-1 to 7-6 are sterilized by autoclaving (moist heat) with F0 > 15 min.

Additionally, a product commercialized by Allergan, Juvederm Volift, is tested as sample 7-7. This product is manufactured using the Vycross crosslinking technology which is known for referring to a crosslinked hyaluronic acid prepared with hyaluronic acids of different mass-average molar mass including a large proportion of LMW HA. Said product comprises lidocaine HC1, a total HA concentration of 17.5 mg/g and is sterilized by moist heat.

TABLE 10

| Samples | Concentration of crosslinked HMW HA, 4.00 MDa (mg/g) | Concentration of uncrosslinked HMW HA, 4.00 MDa (mg/g) | Concentration of uncrosslinked LMW HA, 0.10 MDa (mg/g) | Total sHA (%) | MOD sHA (%) | LMW sHA (0.02 <_ Mw <_ 0.30 MDa) (%) | HMW sHA (Mw > 0.30 MDa) (%) |
|---|---|---|---|---|---|---|---|
| 7-1 (reference) | 10.5 | 0 | 0 | 20.6 | 17 | 15.8 | 4.8 |
| 7-2 (reference) | 0 | 4.5 | 0 | 100 | 0 | 12.5 | 87.5 |
| 7-3 (reference) | 0 | 0 | 10 | 100 | 0 | 96.2 | 0.3 |
| 7-4 (invention) | 10.5 | 0 | 4.5 | 40.5 | 5 | 33.8 | 5.7 |
| 7-5 (invention) | 10.5 | 4.5 | 10 | 58.3 | 3 | 37.0 | 17.7 |
| 7-6 (comparative) | 10.5 | 4.5 | 0 | 36.3 | 9 | 7.7 | 28.4 |
| 7-7 (comparative) | - | - | - | 20.7 | 7 | 15.9 | 2.3 |

Sample 7-1 (100% of HA involved within a crosslinked reaction) shows sHA with a MOD of 17% whereas samples 7-2 and 7-3 (sterilized uncrosslinked HA alone) logically do not present any MOD. The crosslinked sample 7-1 releases sHA fragments (predominantly LMW) which are highly modified with the crosslinker. Sample 7-2 which was manufactured with 4.00 MDa non-crosslinked HA raw material, presents its largest sHA proportion higher than 0.30 MDa whereas sample 7-3 manufactured using 0.10 MDa non-crosslinked HA raw material presents its whole sHA proportion lower than 0.30 MDa.

Supplemented compositions according to the invention have sHA with a lower MOD than compositions prepared without supplementation (samples 7-4 vs 7-1, 7-5 vs 7-6) due to the fact that the uncrosslinked LMW (0.10 MDa) HA increases the molar quantity of HA disaccharide units in the sHA thus decreasing MOD values. It is noteworthy that gels according to the invention (samples 7-3 and 7-4) present a higher proportion of LMW sHA lower than 0.3 MDa compared to the samples 7-5, 7-6, and 7-7. Especially, the commercial sample 7-7 manufactured with a large proportion of non-crosslinked LMW HA for crosslinking does not present such high amount of LMW sHA.

We can theoretically expect that a composition comprising crosslinked HA HMW supplemented with non-crosslinked HA LMW has a total sHA percentage equal to the sum of:
the percentage of non-crosslinked HA LMW with respect to the total mass of HA within the composition and
the percentage of total sHA with respect to the total mass of HA measured for the crosslinked part alone.

Sample 7-1 (100% of HA involved within a crosslinked reaction) shows 20.6 %, of total sHA with respect to the total mass of HA within the composition thus, we can theoretically expect that sample 7-3 (same base of crosslinked HA as sample 7-1 further comprising non-crosslinked HA LMW) has around 30 + 20.6 = 50.6% of sHA with respect to the total mass of HA within the composition.

However, the measured total sHA percentage for sample 7-3 is around 10 points lower (40.5%). This suggests that supplementation according to the invention protects HA chains during manufacturing process lowering the release of LMW sHA, in other words, that it increases the resistance to degradation of the composition.

In conclusion, SEC and ¹H NMR techniques can be successfully used to characterize gel sHA and help distinguishing standard gels from gels according to the present invention.

### Example 8

### Influence of Mass Ratio « HA LMW Non-crosslinked » Over « HA HMW »

Studied samples are presented hereafter.

Sample 8-1, according to the invention, is a gel of crosslinked hyaluronic acid sterilized within syringes by autoclaving (F0 > 15 minutes), with a hyaluronic acid total concentration of 20 mg/g and resulting from the mixing, before sterilization, in a proportion of 1:1 of:
a crosslinked hyaluronic acid gel prepared with non-crosslinked HA HMW (4.00 MDa, HTL, pharmaceutical/medical Eur. Ph. grade) and BDDE (Sigma Aldrich, ≥ 95%), with a hyaluronic acid total concentration of 20 mg/g within a phosphate buffer and a crosslinking rate of 2%; and,
a non-crosslinked hyaluronic acid solution of 20 mg/g of non-crosslinked HA LMW (0.10 MDa, Altergon, pharmaceutical/medical Eur. Ph. grade).

Sample 8-2 is a comparative gel prepared as sample 8-1 but with a non-crosslinked hyaluronic acid solution of 2 mg/g of non-crosslinked HA LMW (010 MDa, Altergon, pharmaceutical/medical Eur. Ph. grade) within a phosphate buffer instead of the non-crosslinked hyaluronic acid solution of 20 mg/g of non-crosslinked HA LMW (0.10 MDa, Altergon, pharmaceutical/medical Eur. Ph. grade).

Sample 8-3 is a reference gel having a crosslinked HMW HA concentration of 10 mg/g with respect of the total mass of the gel. It is prepared as sample 8-1 but using phosphate buffer alone instead of the non-crosslinked hyaluronic acid solution of 20 mg/g of non-crosslinked HA LMW (0.10 MDa, Altergon, pharmaceutical/medical Eur. Ph. grade).

TABLE 11

| Samples | Concentration of crosslinked HMW HA (mg/g) | Concentration of uncrosslinked LMW HA (mg/g) | Mass ratio uncrosslinked LMW HA: crosslinked HMW HA | ΔG′ (%) | G′ improvement (%) |
|---|---|---|---|---|---|
| 8-1 (invention) | 10 | 10 | 1:1 | -17 | 23 |
| 8-2 (comparative) | 10 | 1 | 1:10 | -20 | 9 |
| 8-3 (reference) | 10 | 0 | 0 | -22 | 0 |

Sample 8-1 has a good G′ improvement percentage but sample 8-2 has a lower one. Accordingly, it can be inferred that a sufficient proportion of LMW HA is needed in order to have a significant G′ improvement percentage.

1. A hyaluronic acid-based aqueous gel comprising crosslinked hyaluronic acid, the gel comprises at least:
a water-soluble hyaluronic acid with a mass-average molar mass ranging from 0.02 MDa to 0.30 MDa, called “sHA LMW”, in an amount of from 30% to 70% by mass with respect to the total mass of hyaluronic acid; and
optionally, a water-soluble hyaluronic acid with a mass-average molar mass greater than 0.30 MDa, called “sHA HMW”, and in that said water-soluble hyaluronic acids have a modification degree less than or equal to 5%, respective percentages of soluble hyaluronic acids being determined by diluting the aqueous gel within a pH 7.2 solution of 150 nM sodium nitrate containing 0.02 % NaN₃ at 25° C. for 5 days and centrifugating it at 4400 rpm for 10 minutes and filtering at 0.45 mm such diluted gel to obtain a filtrate then analysing said filtrate with a size exclusion chromatography instrument equipped with a multiangle light scattering (MALS) detector and a refractive index (RI) detector with a refractive index increment (dn/dc) set at 0.165 mL/g and a liquid chromathography pumping station equipped with a dual set of size exclusion columns adapted for molecules with mass-average molar masscomprised from 0.001 Da to 20 MDa, with a pH 7.2 mobile phase of sodium nitrate at a flow rate of 0.3 mL/min.

2. The aqueous gel as claimed in claim 1, wherein the gel is a soft gel having a phase angle (8) between 15 ° and 50 ° and an elastic modulus G′ less than or equal to 150 Pa measured for a stress of 5 Pa at room temperature using a rheometer having a cone/plate geometry applying an oscillation stress sweep at a frequency of 1 Hz and an extrusion force less than or equal to 18 N, measured at a fixed speed of about 12.5 mm/min, in syringes with an internal diameter greater than or equal to 6.3 mm, with a needle of external diameter less than or equal to 0.3 mm (30 G) and length ½”, at room temperature.

3. The aqueous gel as claimed in claim 1, further comprising from 35% to 65% by mass of “sHA LMW” with respect to the total mass of said aqueous gel.

4. The aqueous gel as claimed in claim 1, wherein said water-soluble hyaluronic acid called “sHA LMW” has a mass-average molar mass ranging from 0.04 MDa to 0.20 MDa, .

5. The aqueous gel as claimed in claim 1, wherein said water-soluble hyaluronic acid called “sHA HMW” has a mass-average molar mass greater than 0.30 MDa and lower than or equal to 4.00 MDa.

6. The aqueous gel as claimed in claim 1, further comprising from 10% to 30% by of “sHA HMW”, with respect to the total mass of said aqueous gel.

7. The aqueous gel as claimed in claim 1, wherein said crosslinked hyaluronic acid is present in an amount of at least 5 mg/g with respect to the total mass of said aqueous gel.

8. The aqueous gel as claimed in claim 1, wherein said water-soluble hyaluronic acid called “sHA LMW” is present in an amount of at least 3 mg/g with respect to the total mass of said aqueous gel.

9. The aqueous gel as claimed in claim 1, wherein the total hyaluronic acid concentration is from 10 to 40 mg/g with respect to the total mass of said aqueous gel.

10. The aqueous gel as claimed in claim 1, wherein the modification degree of the total hyaluronic acids is less than or equal to 5% .

11. The aqueous gel as claimed in claim 1, wherein:
the crosslinked hyaluronic acid is present in an amount ranging from 6 mg/g to 12 mg/g with respect to the total mass of the aqueous gel,
the water-soluble hyaluronic acid called “sHA LMW” is present in an amount ranging from 12 mg/g to 20 mg/g with respect to the total mass of the aqueous gel,
the water-soluble hyaluronic acid called “sHA HMW” is present in an amount ranging from 3 mg/g to 6 mg/g with respect to the total mass of the aqueous gel, and
said aqueous gel comprising a total hyaluronic acid concentration of 20 to 30 mg/g with respect to the total mass of the aqueous gel.

12. The aqueous gel as claimed in claim 1, wherein said gel is subjected to a heat sterilization step .

13. A process for preparing an aqueous gel comprising at least one crosslinked hyaluronic acid and at least one non-crosslinked hyaluronic acid, said process comprising at least the steps consisting in:
a) having an aqueous solution comprising at least one crosslinked hyaluronic acid, called “crosslinked HA HMW”, formed from a non-crosslinked hyaluronic acid of mass-average molar mass greater than or equal to 0.50 MDa, and a crosslinking agent:
b) having an aqueous solution comprising at least one non-crosslinked hyaluronic acid, called “non-crosslinked HA LMW”, of mass-average molar mass ranging from 0.04 MDa to 0.30 MDa ; and,
c) forming a homogeneous mixture of all or part of said solutions of steps a) and b),
wherein hyaluronic acid is present in a mass ratio of “non-crosslinked HA LMW” to “HA HMW” of 1:3 to 2:1.

14. The process as claimed in claim 13, wherein said “crosslinked HA HMW” is prepared with a crosslinking ratio of less than or equal to 8% by mass.

15. The process as claimed in claim 13, wherein said crosslinking agent used for the formation of the “crosslinked HA HMW” is a bi- or multi-functional epoxy crosslinking agent .

16. The process as claimed in claim 13, further comprising the use of at least one non-crosslinked hyaluronic acid of mass-average molar mass greater than or equal to 0.50 MDa MDa, called “non-crosslinked HA HMW”.

17. The process as claimed in claim 16, wherein all or part of said “uncrosslinked HA HMW” is used in the aqueous solution of step b).

18. The process as claimed in claim 17, wherein in step c) the aqueous solution of said “uncrosslinked HA HMW” and the aqueous solution of step b) arc mixed concomitantly with the aqueous solution of step a).

19. The process as claimed in claim 13, wherein step c) comprises at least one step of extruding the formed mixture through at least one grid.

20. The process as claimed in claim 13, wherein the aqueous gel comprises at least 5 mg/g of “crosslinked HA HMW” with respect to the total mass of said aqueous gel.

21. The process as claimed in claim 13. wherein the aqueous gel comprises at least 3 mg/g, preferentially 10 mg/g, of said “uncrosslinked HA LMW” with respect to the total mass of said aqueous gel.

22. The process as claimed in claim 13, wherein the aqueous gel has a total hyaluronic acid concentration ranging from 10 to 40 mg/g mg/g with respect to the total mass of said aqueous gel.

23. The process as claimed in claim 13 wherein the aqueous gel comprises the use of hyaluronic acid in a mass ratio of “uncrosslinked HA LMW” to “HA HMW” of 1:3 to 5:3.

24. The process as claimed in claim 13, wherein the aqueous gel has a modification degree of said hyaluronic acids of less than or equal to 5% .

25. The process as claimed in claim 13, wherein the proccss uses:
in step a), an aqueous solution comprising from 5 to 15 mg/g of “crosslinked HA HMW”;
– in step b), an aqueous solution comprising from 50 to 60 mg/g of “uncrosslinked HA LMW” and from 5 to 20 mg/g of “uncrosslinked HA HMW” and in that,
said gel formed has a total hyaluronic acid concentration of 20 to 30 mg/g with respect to its total mass.

26. The process as claimed in claim 13, for preparing a sterile and injectable gel further comprising at least one step of using an aqueous gel obtained according to claim 13 or as defined according to claim 1, and a heat sterilization step .

27. The process as claimed in claim 26, wherein the aqueous gel is packaged in a syringe prior to its sterilization.

28. An aqueous gel of hyaluronic acids obtainable by a process as claimed in claim 13.

29. The aqueous gel according to claim 28, wherein the aqueous gel is a soft gel having a phase angle (δ) between 15° and 50° and an elastic modulus G′ less than or equal to 150 Pa, measured for a stress of 5 Pa at room temperature using a rheometer having a cone/plate geometry applying an oscillation stress sweep at a frequency of 1 Hz and an extrusion force less than or equal to 18 N, preferably less than 15 N, measured at a fixed speed of about 12.5 mm/min, in syringes with an internal diameter greater than or equal to 6.3 mm, with a needle with an external diameter of less than or equal to 0.3 mm (30 G) and with a needle length of ½”, at room temperature.

30. A cosmetic and/or dermatological composition comprising an aqueous gel as claimed in claim 1 or an aqueous gel as claimed in claim 28.

31. A method for preventing and/or treating an alteration in a surface appearance of skin comprising at least one step of topically applying on a subject such as a patient in need thereof or a step of administering by injection into the body of a subject such as a patient in need thereof an effective amount of the aqueous gel as claimed in claim 1 or in claim 28 or the dermatological composition as claimed in claim 30.

32. A method for augmenting and/or filling soft tissues comprising at least one step of topically applying on a subject such as a patient in need thereof or a step of administering by injection into the body of a subject such as a patient in need thereof an effective amount of the aqueous gel as claimed in claim 1 or in claim 28 or the dermatological composition as claimed in claim 30.

33. The non-therapeutic cosmetic use of the aqueous gel as claimed in claim 1 or the aqueous gel as claimed in claim 28 or the cosmetic composition as claimed in claim 30 for preventing and/or treating an alteration in the surface appearance of the skin.

34. The non-therapeutic cosmetic use of the aqueous gel as claimed in claim 1 or the aqueous gel as claimed in claim 28or the cosmetic composition as claimed in claim 30 for augmenting and/or filling soft tissues .

35. A kit comprising a pre-filled syringe comprising the aqueous gel as claimed in claim 1 or the aqueous gel as claimed in claim 28 or the cosmetic and/or dermatological composition as claimed in claim 30 and instructions for use.
